(19) United States
(12) Patent Application Publication
(10) Pub. No.: US 2026/0344631 A1
(43) Pub. Date: Nov. 5, 2026

(54) Genetic Constructs for Localised Population Suppression

(71) Applicant: Imperial College Innovations Limited, London Greater London (GB)

(72) Inventors: Austin Burt, London Greater London (GB); Katie Willis, London Greater London (GB)

(73) Assignee: Imperial College Innovations Limited, London Greater London (GB)

(21) Appl. No.: 19/140,030

(22) PCT Filed: Dec. 15, 2023
(86) PCT No.: PCT/GB2023/053269

§ 371 Date: Jun. 17, 2025

(30) Foreign Application Priority Data
Dec. 19, 2022 (GB) 2219158.9

(51) Int. Cl. A01K 67/68; C12N 15/85

(52) U.S. Cl. CPC A01K 67/68; C12N 15/8509; A01K 2217/072; A01K 2217/075; A01K 2217/20; A01K 2227/706; C12N 2830/34

(57) ABSTRACT

The invention relates to genetic constructs, and in particular to genetic constructs for use in population suppression, and pest control. The invention particularly relates to genetic constructs capable of disrupting a haplo-sufficient gene needed for survival or reproduction in an organism, wherein the genetic construct encodes a genomic editor that creates a dominant lethal or sterile mutation in a target gene of the organism. The invention is also concerned with methods of suppressing wild-type populations by use of the genetic constructs described herein.

The invention relates to genetic constructs, and in particular to genetic constructs for use in population suppression, and pest control. The invention particularly relates to genetic constructs capable of disrupting a haplo-sufficient gene needed for survival or reproduction in an organism, wherein the genetic construct encodes a genomic editor that creates a dominant lethal or sterile mutation in a target gene of the organism. The invention is also concerned with methods of suppressing wild-type populations by use of the genetic constructs described herein.

Currently, pest control is largely based on the use of chemicals. Other alternatives include physical control, including nets or fences, and biological control, such as the introduction of predators. Genetic biocontrol strategies may offer advantages due to their species specificity, ecological friendliness and ability to spread by exploiting natural mate-seeking behaviour of the species. There are several existing genetic control methods. The most widely used approach is the sterile insect technique (SIT). A successful example is the control of the new world screwworm, now implemented through programs such as The Screwworm Barrier Maintenance Program in Panama managed jointly by the U.S. and Panamanian governments. Other applications include the control of the Mediterranean fruitfly, an agricultural pest, implemented by government agencies such as the FOA and IAEA. Release of insects carrying dominant lethal genes (RIDL) is another alternative developed and commercialised by the private company Oxitec. The release of mosquitoes carrying Wolbachia bacteria have been developed for population suppression by companies such as Verily and Mosquito Mate, or for population modification by not-for-profits such as The World Mosquito Program.

As mentioned above, SIT has been used to control some pest populations, however for others, it is not sufficiently efficient to give satisfactory control for a reasonable price. There have been a number of proposals for more efficient localised genetic biocontrol. Y-linked editors (YLEs) and fs-RIDL-Drive constructs, modelled by Burt and Deredec (2018, P. Roy. Soc. Biol. Sci. 285 (1883)), are expected to give efficient and localised control, however, they have some drawbacks which make them difficult to use in some species. YLEs require expression from the Y chromosome, which may be difficult to achieve, and also means they are not applicable for species without a Y chromosome. Additionally, fs-RIDL-Drive requires high homing efficiency, which so far has been difficult to achieve in some species such as mice. For both YLE and fs-RIDL-Drive, a female-specific haplo-insufficient gene is required, which are rare, or in the case of the YLE, an X-linked target site.

For some use cases, genetic control elements would be greatly improved if they could provide non-localised (self-spreading) relatively complete suppression, in which populations over a landscape are reduced to very small numbers from one or a few releases. There have been suggestions for achieving this, including using a driving Y chromosome or using the homing reaction to knock out a gene needed for survival or reproduction, or using a toxin antidote system involving haplo-insufficient genes and rescue copies thereof, however, these may not be appropriate for all species. Additionally, creating a driving Y chromosome has proven to be difficult, and homing may not occur at a sufficiently high rate in many species, and haplo-insufficient genes can be difficult to work with.

For some use cases, genetic control elements would also be improved if they could provide non-localised (self-spreading) partial control. This may be useful if the end goal of the control programme is partial control, or if the end goal is complete control, but there is a desire to approach that in a step-wise manner as part of a risk mitigation strategy. Currently, no strategies have been proposed that give self-spreading partial control when fitness effects and editing rates are idealised (i.e., 0 or 1).

There is, therefore, the need for a genetic control element, which does not require high homing efficiency or the need for a female-specific haplo-insufficient gene, or expression off of a sex chromosome, and which can provide both localised suppression and complete or partial non-localised suppression.

As described in the Examples, the inventors have developed a novel genetic construct, which offers increased efficiency over SIT, RIDL or Wolbachia for population suppression, owing to the ability of the construct to persist in the population over multiple generations. When released without a booster, this genetic construct does not require homing, and no sex-specific gene is necessary and target sites can be autosomal, reducing the constraint on the choice of target sites. The genetic construct of the invention will give efficient suppression of wild populations that, depending on the configuration, may be either localised to the release area or self-spreading to other areas and give either partial or complete suppression. As such, potential applications of the genetic construct include the control of pest populations, which cannot be satisfactorily controlled with current interventions.

Accordingly, in a first aspect of the invention, there is provided a genetic construct comprising a first nucleotide sequence configured to disrupt a haplo-sufficient gene needed for survival or reproduction in a male and/or female organism, or to integrate into or near to a disrupted allele of the haplo-sufficient gene, and a second nucleotide sequence encoding a genomic editor that creates a dominant lethal or sterile mutation in a target gene expressed in the male and/or female organism, such that a homozygote for the genetic construct is lethal or sterile and/or a heterozygote for the dominant lethal or sterile mutation is lethal or sterile.

In some embodiments, there is provided a genetic construct comprising a first nucleotide sequence configured to disrupt a haplo-sufficient gene needed for survival or reproduction in a male and/or female organism, and a second nucleotide sequence encoding a genomic editor that creates a dominant lethal or sterile mutation in a target gene expressed in the male and/or female organism, such that a homozygote for the genetic construct is lethal or sterile and/or a heterozygote for the dominant lethal or sterile mutation is lethal or sterile.

The inventors have surprisingly identified that the genetic construct according to the invention can be inserted into a haplo-sufficient gene of members of a species and released into a population, in order to suppress the population in an efficient manner over a period of time. Advantageously, and preferably, the inventors discovered that depending on the precise configuration of embodiments of the construct, the genetic construct can have three different types of potentially useful impact when released into a target population: localised suppression, non-localised partial suppression, or non-localised complete suppression. These different impacts are hugely beneficial depending on the species of organism (i.e. pest) whose population is being suppressed.

It is well-known to the skilled person that a gene is a haplo-sufficient (HS) gene if only one working copy of the gene is sufficient to maintain a normal expression and function of the gene. This is because the functional allele of a haplo-sufficient gene is dominant, whilst the non-functional allele of a haplo-sufficient gene is recessive. However, haplo-insufficient (HI) genes are those where one working copy of the gene is not sufficient to maintain a normal expression and function of the gene. This is because the non-functional allele of a haplo-insufficient gene is effectively dominant.

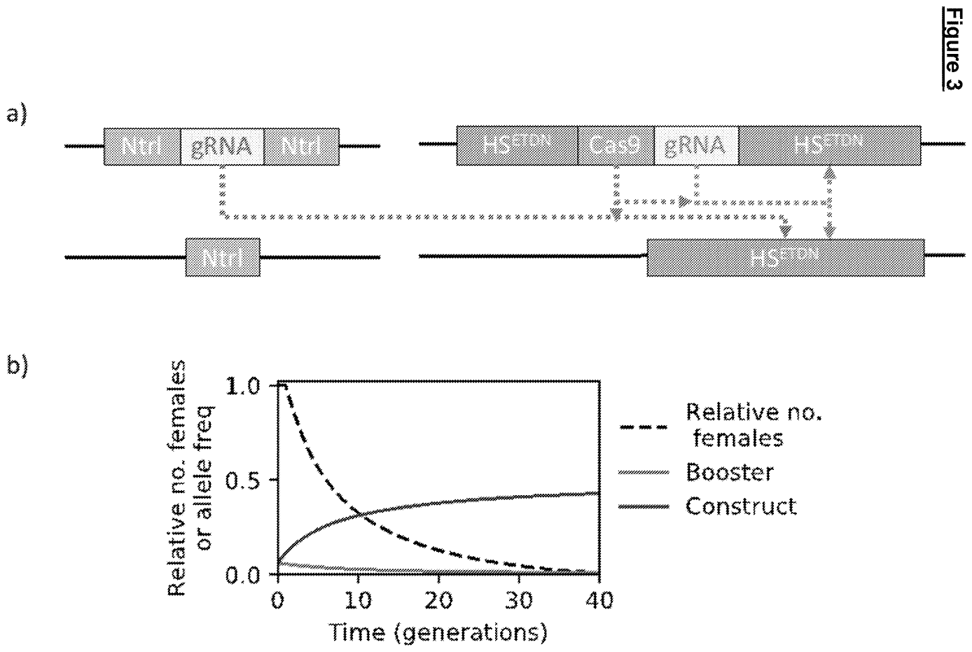

As illustrated in FIG. 2a, the haplo-sufficient gene (HS) is disrupted by the insertion of the genetic construct (shown for illustration purposes as Cas9 and gRNA). Accordingly, the genetic construct may be configured to disrupt the haplo-sufficient gene via integration of the genetic construct into the haplo-sufficient gene at an integration site or disruption site. For example, in one embodiment, the genetic construct may introduce a premature stop codon into the haplo-sufficient gene. Alternatively, the genetic construct may disrupt the haplo-sufficient gene by making a change to the gene, such as deletion of the gene, which may be deletion of all of the gene, or part of the gene.

The genetic construct may integrate into the centre of the haplo-sufficient gene. The construct may integrate into a region of the haplo-sufficient gene between its 5′ promoter and 3′ terminus. The construct may integrate between exons or introns of the haplo-sufficient gene.

However, in some embodiments, the genetic construct may be configured to integrate into a disrupted allele of the haplo-sufficient gene. Accordingly, the disruption of the haplo-sufficient gene may be caused by means other than the integration of the genetic construct. Preferably, in this embodiment, the disruption is caused by the introduction of a knock-out mutation, preferably wherein the knockout mutation is caused by the introduction of a premature stop codon, optionally by insertion of a sequence. Alternatively, the knock-out mutation may be caused by the deletion of part or all of the haplo-sufficient gene (FIG. 2d).

In some embodiments, the genetic construct may be configured to integrate at a location outside of, but near to, a disrupted allele of the haplo-sufficient gene. Preferably, in this embodiment, the genetic construct is configured to create a separate modification that disrupts the haplo-sufficient gene. Accordingly, in one embodiment, the genetic construct integrates at a location near to the disrupted allele of the haplo-sufficient gene, preferably within 10, 8, 6, 4, 2, or 1 centiMorgan of the disrupted allele of the haplo-sufficient gene, and more preferably within 1 centiMorgan of the disrupted allele of the haplo-sufficient gene (FIG. 2d).

An important feature of this new method of population suppression, is that the construct is associated with recessive disruption of the haplo-sufficient gene. One way to achieve this association is for the construct to be causally responsible for the disruption, but that is not the only way, and there are other ways of achieving this, including making two separate manipulations in close proximity, one to disrupt the haplo-sufficient gene and the other to integrate the construct. These two manipulations may be done in either order. Thus, references to “the haplo-sufficient gene disrupted by the construct” and similar phrases, include both causal and non-causal associations between the disrupted haplo-sufficient gene and the construct.

Preferably, the gene into or near to which the genetic construct integrates, and which is disrupted or the construct disrupts, is haplo-sufficient, and so organisms that are heterozygous for one wild type allele and one disrupted allele, in an otherwise wild type genetic background, have normal or near normal fitness.

In one embodiment, the genetic construct may preferably result in localised suppression of a population, i.e. it will be geographically localised to the release area. In order to achieve localised suppression, both male and female homozygotes for the genetic construct are preferably inviable or sterile. Accordingly, in this embodiment, the genetic construct is configured to preferably disrupt a haplo-sufficient gene needed for survival or reproduction in a male and female organism, or to integrate into or near to a disrupted allele of the haplo-sufficient gene, such that male and female homozygotes for the genetic construct are lethal or sterile. Preferably, when the genetic construct is integrated near to a disrupted allele of the haplo-sufficient gene, the genetic construct is integrated within 10, 8, 6, 4, 2, or 1 centiMorgan of the disrupted allele of the haplo-sufficient gene, and more preferably within 1 centiMorgan of the disrupted allele of the haplo-sufficient gene.

Additionally, for localised suppression, the genomic editor preferably creates a dominant lethal or sterile phenotype in female organisms or in both sexes. Accordingly, in this embodiment, the genomic editor preferably creates a dominant lethal or sterile mutation in a target gene expressed in the female organism, or in a target gene expressed in the male and female organism.

Accordingly, in this localised suppression embodiment, the presence of the genetic construct in or near to the haplo-sufficient gene is associated with bi-sex recessive lethality or sterility, i.e. a recessive lethal or sterile phenotype in both males and females. Preferably, the recessive lethality or sterility is caused by introducing a premature stop codon into the haplo-sufficient gene or by deletion of all or part of the gene. In a preferred embodiment, the genomic editor creates a bi-sex or female-specific dominant mutation, or a female-specific dominant mutation with male-specific recessive lethality.

The inventors have generated three molecular configurations for the genetic constructs that are capable of achieving localised population suppression, and these are illustrated in FIGS. 2a-c.

As shown in FIG. 2a, in one configuration, the genomic editor encoded by the nucleotide sequence of the genetic construct targets a site in a wild type allele of the same haplo-sufficient gene that is disrupted by the integration of the genetic construct. Accordingly, in one embodiment, the target gene is a wild type allele of the disrupted haplo-sufficient gene.

Preferably, the genomic editor creates a dominant negative or a dominant gain of function mutation in a wild-type allele of the target gene. Accordingly, in a preferred embodiment, the genomic editor creates a dominant negative or a dominant gain of function mutation in a wild type allele of the disrupted haplo-sufficient gene.

Preferably, the genomic editor targets a site in the target gene (i.e. the wild type allele of the haplo-sufficient gene) that is downstream of the integration site. Alternatively, the genomic editor may target a site in the haplo-sufficient gene that is upstream of the integration site. In an embodiment in which the target site of the genomic editor is upstream of the integration site, the target site on the chromosome containing the genetic construct is preferably recoded or removed, such that the chromosome is protected from the dominant mutation.

Preferably, the genomic editor creates a dominant negative or a dominant gain of function mutation in a female-specific exon of a haplo-sufficient gene needed in a male and female organism, optionally wherein the haplo-sufficient gene is a homolog of the Drosophila gene doublesex or fruitless.

Preferably, the chromosome comprising the construct is not edited, because the target site has been modified or removed, or, if it is edited, the dominant negative or dominant gain of function mutation is not expressed, due to the presence of the genetic construct in the haplo-sufficient gene, such that organisms comprising one copy of the genetic construct and one wild-type allele, have normal or near normal fitness. However, preferably organisms that are homozygous for the genetic construct are inviable or sterile. Moreover, preferably organisms that are heterozygous for the genetic construct and the dominant negative or dominant gain of function mutation are inviable or sterile, either if female or regardless of sex. Preferably, the heterozygote for the dominant lethal or sterile mutation is lethal or sterile when inherited in the absence of the genetic construct.

Alternatively, as illustrated in FIG. 2b, in another configuration, the genomic editor encoded by the nucleotide sequence of the genetic construct targets a gene that is closely linked to the haplo-sufficient gene in which or near to which the genetic construct integrates. Accordingly, in one embodiment, the target gene is a gene that is preferably located near to the disrupted haplo-sufficient gene (i.e. the integration site of the genetic construct). Therefore, in a preferred embodiment, the genomic editor preferably creates a dominant negative or a dominant gain of function mutation in a wild type allele of a target gene that is located near to the disrupted haplo-sufficient gene. Alternatively, in another preferred embodiment, the genomic editor creates a knock-out mutation in a wild type allele of a haplo-insufficient target gene that is located near to the disrupted haplo-sufficient gene.

Preferably, when the target gene and the disrupted haplo-sufficient gene (i.e. the integration site of the genetic construct) are located near to one another, they comprise a meiotic recombination fraction of less than 5%, less than 4%, less than 3%, less than 2%, or less than 1%. Most preferably, the target gene and the disrupted haplo-sufficient gene comprise a meiotic recombination fraction of less than 1%.

Preferably, the target gene and the disrupted haplo-sufficient gene are inverted. Advantageously, this reduces the frequency of recombination between the two genes.

Preferably, the copy of the target gene on the same chromosome as the genetic construct is modified to be resistant to the genomic editor. For example, if the genomic editor creates a dominant negative mutation in a haplo-sufficient gene, then preferably the resistant chromosome comprises a deletion of the target site or a deletion of the entire target gene. Alternatively, if the genomic editor creates a knockout mutation in a haplo-insufficient gene, then preferably the resistant chromosome comprises a recoded version of the target site, such that it is functional but not recognised by the editor. In this case, preferably, the genomic editor does not stimulate homologous recombinational repair.

Alternatively, as illustrated in FIG. 2c, in another configuration, the genomic editor encoded by the nucleotide sequence of the genetic construct may target a gene that is distantly linked to the haplo-sufficient gene in which or near to which the genetic construct integrates. Accordingly, in one embodiment, the target gene is a gene that is preferably distantly located from the disrupted haplo-sufficient gene (i.e. the integration site of the genetic construct). Therefore, in a preferred embodiment, the genomic editor creates a dominant negative or a dominant gain of function mutation in a wild type allele of a target gene that is distantly located from the disrupted haplo-sufficient gene. Alternatively, in another preferred embodiment, the genomic editor creates a knock-out mutation in a wild type allele of a haplo-insufficient target gene that is distantly located from the disrupted haplo-sufficient gene.

Preferably, when the target gene and the disrupted haplo-sufficient gene (i.e. the integration site of the genetic construct) are distantly located from one another, they comprise a meiotic recombination fraction of greater than 0.5%, greater than 1%, greater than 2%, greater than 3%, greater than 4%, or greater than 5%. Most preferably, the target gene and the disrupted haplo-sufficient gene comprise a meiotic recombination fraction of greater than 1%. In one embodiment, the target gene is located on a different chromosome from the disrupted haplo-sufficient gene. In another embodiment, the target gene is located on the same chromosome as the disrupted haplo-sufficient gene.

As shown in FIG. 2c, the genetic construct may further comprise a rescue copy of the target gene that is not recognised by the genomic editor. Accordingly, in one embodiment, the genetic construct further comprises a nucleotide sequence encoding a rescue copy of the target gene. The nucleotide sequence encoding the rescue copy of the target gene is not recognised by the genomic editor. For example, in an embodiment in which the genomic editor is a CRISPR/Cas9-based genomic editor, the nucleotide sequence encoding the rescue copy of the target gene differs from the wild-type nucleotide sequence recognised by a gRNA of the genomic editor, such that the rescue copy is not edited by the Caso protein of the genomic editor.

However, the nucleotide sequence encoding the rescue copy of the target gene may also be designed such that it encodes the same amino acid sequence as the wild-type nucleotide sequence, or a functionally equivalent amino acid sequence. In an embodiment in which the genomic editor produces a dominant negative mutation, then preferably the rescue copy on the construct is configured to dilute out the effect of the mutation. Preferably, therefore, the nucleotide sequence encoding the rescue copy of the target gene is configured to have higher expression levels, to achieve this dilution effect. Alternatively, in an embodiment in which the genomic editor produces a knock-out of a haplo-insufficient gene, then preferably the rescue copy comprises a nucleotide sequence encoding the knocked-out protein. In either case, fitness is rescued if there is one edited allele, but not if there are two edited alleles, so that organisms that are heterozygous for the construct and have one edited allele have normal or near normal fitness, whereas organisms that are heterozygous for the construct and have two edited alleles would suffer the consequences of the mutation.

In another embodiment, the genetic construct preferably results in non-localised (self-spreading) complete suppression, in which populations are reduced to very small numbers over a landscape. In order to achieve non-localised complete suppression, homozygotes for the genetic construct are preferably inviable or sterile in only one sex (i.e. males or females). Accordingly, in this embodiment, the genetic construct preferably is configured to disrupt a haplo-sufficient gene needed for survival or reproduction in a male or female organism, or to integrate into or near to a disrupted allele of the haplo-sufficient gene, such that male or female homozygotes for the genetic construct are lethal or sterile. Preferably, when the genetic construct is integrated near to a disrupted allele of the haplo-sufficient gene, the genetic construct is integrated within 10, 8, 6, 4, 2, or 1 centiMorgan of the disrupted allele of the haplo-sufficient gene, and more preferably within 1 centiMorgan of the disrupted allele of the haplo-sufficient gene. Preferably, the genetic construct comprises a nucleotide sequence encoding a genomic editor that creates a dominant lethal or sterile mutation in a target gene expressed in the male and/or female organism.

As illustrated in FIG. 6, this non-localised complete suppression can be achieved in a number of ways, for example, by inserting the construct into genes required only in one sex, or into exons that are required only in one sex (due to sex-specific splicing), by incorporating sequences into the construct that ensure it is spliced out in one sex but not the other, by the genomic editor targeting genes or exons that are needed in one or both sexes, and/or by modifying control regions on a gene so that it is expressed only in one sex, or to increase expression to give partial (sex-specific) protection.

As described for localised suppression, the genomic editor may target the same haplo-sufficient gene into which the genetic construct integrates. Accordingly, in one embodiment, the target gene is a wild type allele of the disrupted haplo-sufficient gene. Preferably, the genomic editor creates a dominant negative or a dominant gain of function mutation in a wild type allele of the target gene. Accordingly, in a preferred embodiment, the genomic editor creates a dominant negative or a dominant gain of function mutation in a wild type allele of the disrupted haplo-sufficient gene.

In another embodiment, the genomic editor may create a dominant negative or a dominant gain of function mutation in a wild type allele of a target gene that is located near to the disrupted haplo-sufficient gene. Alternatively, in another preferred embodiment, the genomic editor creates a knock-out mutation in a wild type allele of a haplo-insufficient target gene that is located near to the disrupted haplo-sufficient gene. Preferably, when the target gene and the disrupted haplo-sufficient gene (i.e. the integration site of the genetic construct), or haplo-insufficient gene, are located near to one another, they comprise a meiotic recombination fraction of less than 5%, less than 4%, less than 3%, less than 2%, or less than 1%. Most preferably, the target gene and the disrupted haplo-sufficient gene comprise a meiotic recombination fraction of less than 1%.

Preferably, the chromosome comprising the genetic construct is not affected by the mutation because the target site of the genomic editor is absent. Alternatively, the mutation created by the genomic editor is not expressed due to the presence of the genetic construct or other mutations at the target site.

As illustrated in FIG. 6a (left-hand side), one way of achieving non-localised complete suppression is for the genetic construct to integrate into and disrupt a female-specific haplo-sufficient gene. Accordingly, in one embodiment, the haplo-sufficient gene is preferably a female-specific haplo-sufficient gene. Preferably, the genomic editor creates a dominant negative mutation or a dominant gain of function mutation in the female-specific haplo-sufficient gene. Examples of female-specific haplo-sufficient genes for which dominant sterile mutations have been observed include but are not limited to homologs of the Drosophila genes ovo, dorsal, torso, easter, and Toll.

In another embodiment, the haplo-sufficient gene may be a male-specific haplo-sufficient gene. In this embodiment, the genomic editor creates a dominant negative mutation or a dominant gain of function mutation in the male-specific haplo-sufficient gene. Examples of male-specific haplo-sufficient genes for which dominant sterile mutations have been observed include, but are not limited to, homologs of the Drosophila genes betaTub85D and whirligig.

Alternatively, the disrupted haplo-sufficient gene may be expressed in both male and female organisms. As such, in order to ensure that the presence of the construct only disrupts the function of the gene in one sex, the genetic construct may integrate into an intron that is spliced in a sex-specific manner, or contain sequences that ensure the construct is spliced out in a sex-specific manner. As such, while the gene as a whole is essential for both sexes, the presence of the construct only disrupts the function of the gene in one sex.

For example, as shown in FIG. 6a (right) and FIG. 6b (left), the genetic construct integrates into and disrupts a female-specific exon of a haplo-sufficient gene needed in both sexes. Therefore, in one embodiment, the disrupted haplo-sufficient gene is disrupted at a female-specific exon of the haplo-sufficient gene. Two examples of genes comprising a female-specific exon are the doublesex (dsx) and fruitless (fru) genes, which may be from the Drosophila species. Accordingly, in one embodiment, the disrupted haplo-sufficient gene is disrupted at a female-specific exon of the doublesex or fruitless genes, or a female-specific exon of a homolog of the doublesex or fruitless genes. Alternatively, in another embodiment, the disrupted haplo-sufficient gene is disrupted at a male-specific exon of the haplo-sufficient gene. Examples of genes comprising a male-specific exon include but are not limited to homologs of the Drosophila genes transformer (tra) and doublesex (dsx). Accordingly, in one embodiment, the disrupted haplo-sufficient gene is disrupted at a male-specific exon of the doublesex or transformer genes, or a male-specific exon of a homolog of the doublesex or transformer genes.

As shown in FIG. 6b (right), the disrupted haplo-sufficient gene is expressed in male and female organisms, and the genetic construct contains sequences that ensure the construct is spliced out in a sex-specific manner. This can be engineered using control sequences that specify the sex-specific splicing of native genes in the target species, including but not limited to homologs of tra, dsx and fru. Accordingly, in one embodiment, the genetic construct comprises a nucleotide sequence that ensures the construct will be spliced out at the RNA in a male organism. In a preferred embodiment, the genetic construct comprises a nucleotide sequence encoding a splicing control sequence of a homolog of a transformer, doublesex or fruitless gene, that ensures the construct will be spliced out at the RNA in a male organism. As such, gene function is predominantly disrupted in females. Alternatively, the genetic construct comprises a nucleotide sequence that ensures the construct will be spliced out at the RNA in a female organism. This can be engineered by using sequences that direct female-specific splicing, such as homologs of the Drosophila transformer gene (Fu et al. 2007 Nat. Biotechnol. 25:353-7). Accordingly, in a preferred embodiment, the genetic construct comprises a nucleotide sequence encoding a splicing control sequence of a homolog of a Drosophila transformer gene, that ensures the construct will be spliced out at the RNA in a female organism. As such, gene function is predominantly disrupted in males. In this embodiment, the mutation produced by the genomic editor is preferably a bi-sex dominant negative mutation. Preferably, the chromosome comprising the genetic construct is protected from the genomic editor because the target site has been recoded.

FIGS. 6c and 6d show examples of how self-spreading complete suppression can also be achieved by inserting the construct to disrupt one gene and have the genomic editor target another gene nearby (<1% recombination), where the target site on the chromosome with the construct has been modified to no longer be recognised by the genomic editor. Accordingly, in one embodiment, the genomic editor may create a mutation in a wild type allele of a target gene that is located near to the disrupted haplo-sufficient gene, preferably wherein the chromosome comprising the genetic construct is protected from the genomic editor because the target site has been recoded. The gene disruption may affect only homozygotes of one sex and the genomic editor may make dominant lethal or sterile mutations that affect both sexes (FIG. 6c). Preferably, therefore, the genetic construct is configured to disrupt a haplo-sufficient gene needed for survival or reproduction in a male or female organism, or to integrate into or near to a disrupted allele of the haplo-sufficient gene, and the genomic editor is configured to create a dominant lethal or sterile mutation in a target gene expressed in the male and female organism. Alternatively, the gene disruption may affect homozygotes of both sexes, and the genomic editor may also affect both sexes, but for heterozygotes with the construct and the mutation, only one sex is affected, which can be achieved by sex-specific enhanced expression of the target gene which, in the heterozygote, nullifies the effect of the mutation in a sex-specific manner (FIG. 6d). As such, non-localised suppression may be achieved by modifying the chromosome containing the construct such that the target gene is recoded and is therefore not recognised by the genomic editor, yet is functional, and also contains control regions so that its expression is enhanced only in one sex, to give partial (sex-specific) protection. Alternatively the chromosome containing the construct can be modified to contain a second copy of the recoded target gene which contains control regions such that it is only expressed in one sex, again to give partial (sex-specific) protection. Accordingly, in one embodiment, the genetic construct is configured to disrupt a haplo-sufficient gene needed for survival or reproduction in a male and female organism, or to integrate into or near to a disrupted allele of the haplo-sufficient gene and the genomic editor is configured to create a dominant lethal or sterile mutation in a target gene expressed in the male and female organism. Preferably, in this embodiment the chromosome comprising the genetic construct is protected from the genomic editor because the target site has been recoded and its expression is enhanced in a sex-specific manner, such that only a male or female heterozygote comprising the construct and the mutation is affected. Alternatively, the chromosome comprising the genetic construct contains a second copy of the recoded target gene with control sequences which ensure it is only expressed in a male or female.

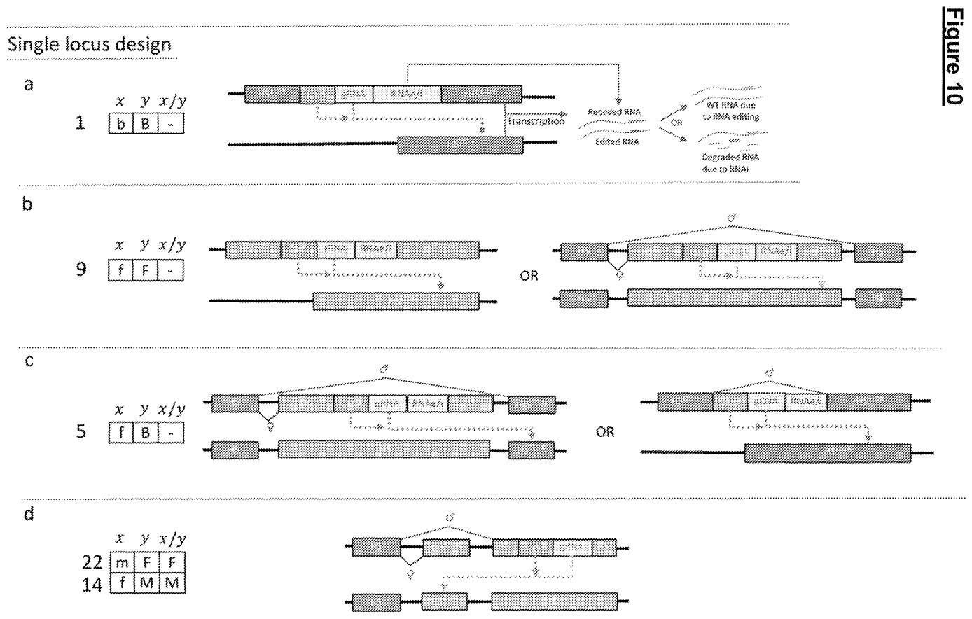

In another configuration, the genetic construct may result in non-localised (self-spreading) partial suppression (FIG. 8). In order to achieve non-localised partial suppression, the genetic construct is preferably recessive sterile or lethal in both sexes or in one sex only. Accordingly, in this embodiment, the genetic construct preferably is configured to disrupt a haplo-sufficient gene needed for survival or reproduction in a male and/or female organism, or to integrate into or near to a disrupted allele of the haplo-sufficient gene, such that male and/or female homozygotes for the genetic construct are lethal or sterile. Additionally, the genetic construct comprises a nucleotide sequence encoding a genomic editor that creates a dominant lethal or sterile mutation in a target gene expressed in the male and/or female organism.

If the genetic construct is bisex (i.e. is associated with disruption of a haplo-sufficient gene essential for fertility or viability in both male and female organisms), then the construct preferably provides dominant protection against the mutation regardless of its location relative to the integration site of the construct (i.e. whether the mutation is in the same gene as is disrupted by the construct, a linked gene or an unlinked gene). One way of achieving this is to preferably encode, in the genetic construct, functions that nullify the effect of the mutation at the RNA level, e.g. by RNA interference (RNAi) or RNA editing (FIG. 8. strategies 1-4). Accordingly, in one embodiment, the genetic construct is inserted into and disrupts a haplo-sufficient gene needed in both males and females, or integrates into or near to a disrupted allele of the haplo-sufficient gene needed in both males and females, and the genomic editor creates a bisex dominant negative mutation. In this embodiment, the genetic construct comprises a nucleotide sequence encoding an RNA editor or an RNA interference module. Preferably, the RNA editor or RNA interference module restores RNA expressed from the mutated gene back to the wild type sequence, by RNA editing, or removes the RNA transcribed from the mutated gene by RNA interference (RNAi). This provides dominant protection against the mutation.

If the genetic construct is sex-specific (i.e. is associated with disruption of a haplo-sufficient gene essential for fertility or viability in either males or females), partial suppression may be achieved if the construct encodes a genomic editor that produces a dominant negative or a dominant gain of function mutation in the wild type allele of the same locus. Accordingly, in one embodiment, the genetic construct is preferably inserted into and disrupts a haplo-sufficient gene needed in a female organism, or integrates into or near to a disrupted allele of the haplo-sufficient gene needed in a female organism, and the genomic editor creates a female-specific dominant negative mutation. Alternatively, the genetic construct is preferably inserted into and disrupts a haplo-sufficient gene needed in a male organism, or integrates into or near to a disrupted allele of the haplo-sufficient gene needed in a male organism, and the genomic editor creates a male-specific dominant negative mutation. Alternatively, in another embodiment, the disrupted haplo-sufficient gene is disrupted at a female-specific exon of the haplo-sufficient gene needed in both sexes, and the genomic editor creates a female-specific or bi-sex dominant negative or gain of function mutation. Alternatively, the disrupted haplo-sufficient gene is disrupted at a male-specific exon of the haplo-sufficient gene needed in both sexes, and the genomic editor creates a male-specific or bi-sex dominant negative or gain of function mutation. Alternatively, in another embodiment, the genetic construct is preferably inserted into a haplo-sufficient gene needed in both males and females, or integrates into or near to a disrupted allele of the haplo-sufficient gene needed in both males and females, but the disrupted haplo-sufficient gene is either disrupted at a female-specific exon, or the construct comprises sequences that ensure it will be spliced out in males, with the result that gene function is predominantly disrupted in females. The genomic editor preferably produces bi-sex dominant negative mutations. Alternatively, the genetic construct is preferably inserted into a haplo-sufficient gene needed in both males and females, or integrates into or near to a disrupted allele of the haplo-sufficient gene needed in both males and females, but the disrupted haplo-sufficient gene is either disrupted at a male-specific exon, or the construct comprises sequences that ensure it will be spliced out in females, with the result that gene function is predominantly disrupted in males. The genomic editor preferably produces female-specific dominant negative mutations, preferably by targeting a female-specific exon of the disrupted haplo-sufficient gene. Preferably, in some embodiments, the construct provides dominant protection against the mutation regardless of whether the mutation is in cis or in trans (i.e. located in the haplo-sufficient gene in which the construct integrates, or the homologous chromosome). Again, this can be achieved if the construct comprises a nucleotide sequence encoding an RNA editor or an RNAi module (FIG. 8. strategies 5-13). In one preferred embodiment, the RNA editor or the RNAi module are expressed only in one sex (FIG. 8. strategies 15 and 21). In another preferred embodiment, the target site on the chromosome containing the construct is modified or removed, such that it is not recognised by the genomic editor.

Alternatively, partial suppression may be achieved if the genetic construct is inserted into a haplo-sufficient gene needed in females, or needed in both males and females, or integrates into or near to a disrupted allele of the haplo-sufficient gene, and the construct comprises sequences that ensure it will be spliced out in males, with the result that gene function is predominantly disrupted in females. The genomic editor preferably creates a dominant negative mutation, a dominant gain of function mutation, or a knock-out mutation in a wild type allele of a target gene that is located near to the disrupted haplo-sufficient gene. Alternatively, partial suppression may be achieved if the genetic construct is inserted into a haplo-sufficient gene needed in males, or needed in both males and females, or integrates into or near to a disrupted allele of the haplo-sufficient gene, and the construct comprises sequences that ensure it will be spliced out in females, with the result that gene function is predominantly disrupted in males. The genomic editor preferably creates a dominant negative mutation, a dominant gain of function mutation, or a knock-out mutation in a wild type allele of a target gene that is located near to the disrupted haplo-sufficient gene. Preferably, the construct comprises a nucleotide sequence encoding an RNA editor or an RNAi module.

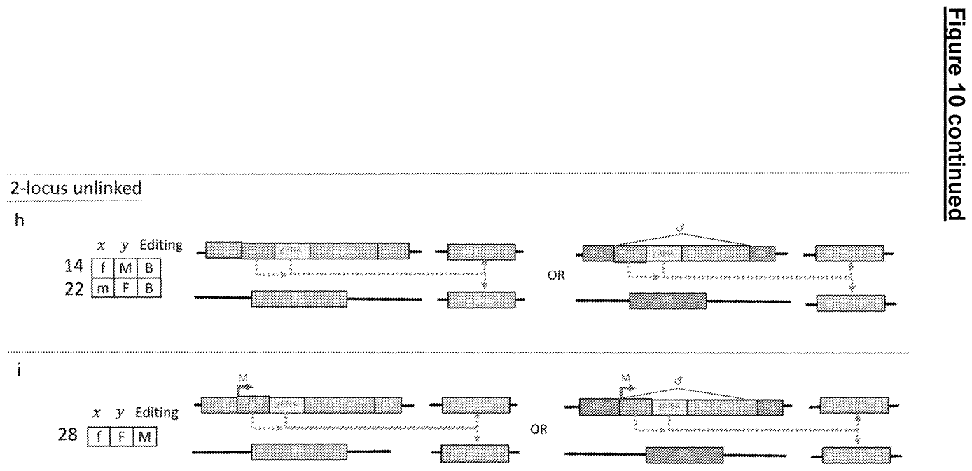

Alternatively, partial suppression may be achieved if the genetic construct is sex-specific (i.e. is associated with the disruption of a haplo-sufficient gene essential for fertility or viability in either males or females), and encodes a genomic editor that produces a dominant negative or a dominant gain of function mutation in the wild type allele of the same locus that affects the opposite sex to the gene disruption (FIG. 8. strategies 14, 18, 22 and 23). This can be achieved by targeting a gene with sex-specific exons for both sexes, such as doublesex, and preferably, the target site on the chromosome with the construct is modified such that it is still functional but no longer recognised by the editor. For example, in a preferred embodiment, the disrupted haplo-sufficient gene is disrupted at a male-specific exon causing recessive male-specific lethality or sterility, and the genomic editor creates a dominant female-specific lethal or sterile mutation in a female-specific exon (FIG. 8. strategy 22). Alternatively, the disrupted haplo-sufficient gene is disrupted at a female-specific exon causing recessive female-specific lethality or sterility, and the genomic editor creates a dominant male-specific lethal or sterile mutation in a male-specific exon. Preferably, the disrupted and target gene is a homolog of doublesex. Preferably, the chromosome comprising the genetic construct is protected from the genomic editor because the target site has been recoded. FIG. 10d illustrates an example molecular design for strategy 22 using a gene such as doublesex.

Alternatively, partial suppression may be achieved if the genetic construct is sex-specific (i.e. is associated with the disruption of a haplo-sufficient gene essential for fertility or viability in either males or females), and encodes a genomic editor that targets a wild type allele of a target gene that is located near to the disrupted haplo-sufficient gene (<1% meiotic recombination), to produce a dominant negative mutation, a gain of function mutation, or a knock-out of a haplo-insufficient gene. Preferably, the genetic construct also provides dominant protection against the mutation regardless of whether the mutation is in cis or in trans (FIG. 8. strategies 5-13). Preferably, the target site on the chromosome containing the construct has been modified or removed such that it is not recognised by the genomic editor. In a preferred embodiment, the RNA editor or RNAi module are expressed only in one sex (FIG. 8. strategies 15 and 21). In another preferred embodiment, the target gene and the disrupted haplo-sufficient gene are inverted, to reduce the frequency of recombination.

Alternatively, partial suppression may be achieved if the genetic construct is sex-specific (i.e. is associated with the disruption of a haplo-sufficient gene essential for fertility or viability in either males or females), and wherein the genetic construct comprises a nucleotide sequence encoding a genomic editor that targets a wild type allele of another gene that is located near to the disrupted haplo-sufficient gene (<1% meiotic recombination), to produce a dominant negative mutation, a gain of function mutation, or a knock-out of a haplo-insufficient gene that affects the opposite sex to the gene disruption. Preferably, the target site on the chromosome containing the construct is modified or removed such that it is no longer recognised by the editor (FIG. 8. strategies 14, 18, 22 and 23). Preferably, the genetic construct comprises sequences that ensure it will be spliced out in a sex-specific manner. Preferably, the target gene and the disrupted haplo-sufficient gene are inverted, to reduce the frequency of recombination.

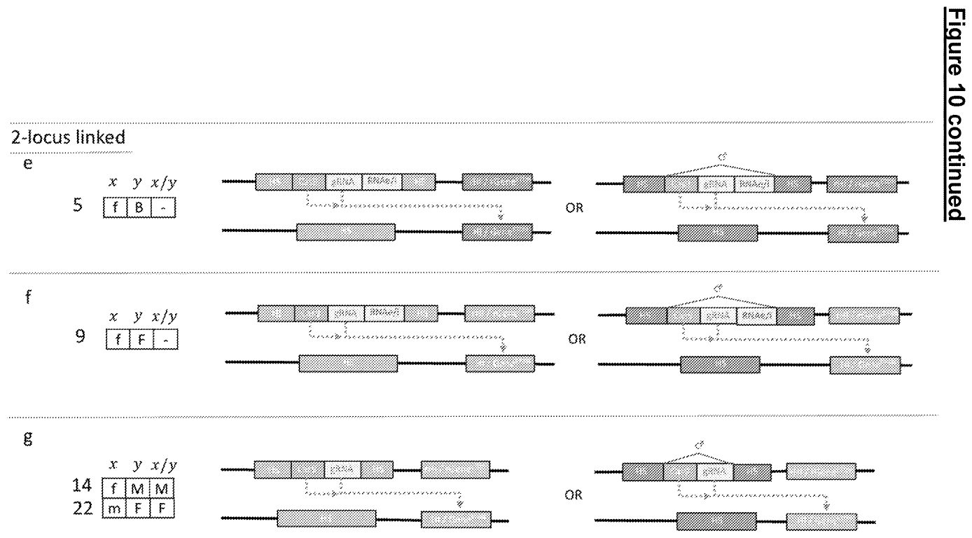

Alternatively, partial suppression may be achieved if the genetic construct is sex-specific (i.e. is associated with the disruption of a haplo-sufficient gene essential for fertility or viability in either males or females), and encodes a genomic editor that targets a wild type allele of another gene that is distantly located from the disrupted gene (>1% meiotic recombination), to produce a dominant negative mutation, a gain of function mutation or knock-out of a haplo-insufficient gene which affects the opposite sex to that affected by the disrupted gene (FIG. 8. strategies 14 and 22, and FIG. 9 strategies 27, 29, 31 and 32). Alternatively, the genomic editor creates a mutation in the opposite sex to that affected by the disruption (FIG. 8. strategies 28 and 30). Preferably, the genetic construct comprises a nucleotide sequence encoding a recoded version of the edited gene, such that it is resistant to the genomic editor whilst also restoring function in individuals carrying a single copy of the dominant edit. Preferably, the genetic construct comprises sequences that ensure it will be spliced out in a sex-specific manner.

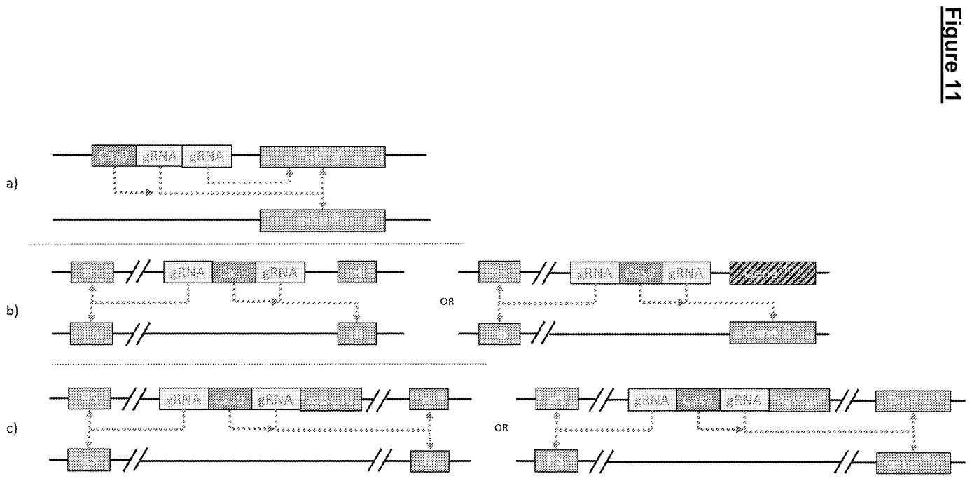

The inventors have generated further molecular configurations for the genetic constructs that are capable of achieving localised population suppression, non-localised (self-spreading) complete suppression, and non-localised (self-spreading) partial suppression, and these are illustrated in FIG. 11.

As illustrated in FIG. 11, the construct is configured to integrate into a location outside of (either near to or more distantly linked to) a haplo-sufficient gene, and designed to induce mutations in the haplo-sufficient gene which cause recessive sterility or lethality, by, for example, including within the construct a second gRNA targeting the haplo-sufficient gene.

Accordingly, in a second aspect of the invention, there is provided a genetic construct comprising a first nucleotide sequence configured to integrate into a location outside of a haplo-sufficient gene needed for survival or reproduction in a male and/or female organism, and a second nucleotide sequence encoding: (i) a first genomic editor that creates a dominant lethal or sterile mutation in a target gene expressed in the male and/or female organism, such that a heterozygote for the dominant lethal or sterile mutation is lethal or sterile; and (ii) a second genomic editor that creates a recessive lethal or sterile mutation in the haplo-sufficient gene, such that a homozygote for the recessive lethal or sterile mutation is lethal or sterile.

Preferably, the heterozygote for the dominant lethal or sterile mutation is lethal or sterile when inherited in the absence of the genetic construct.

A location outside of a haplo-sufficient gene may be a location that is near to or distantly linked to the haplo-sufficient gene. A location that is near to the haplo-sufficient gene may be one which is within 10, 8, 6, 4, 2, or 1 centiMorgan of the haplo-sufficient gene, and more preferably within 1 centiMorgan of the haplo-sufficient gene. Alternatively, a location that is outside of a haplo-sufficient gene may be one which is located on a different chromosome to the haplo-sufficient gene (i.e. unlinked).

Preferably, the haplo-sufficient gene is located downstream of the integration site of the genetic construct. Alternatively, the haplo-sufficient gene may be located upstream of the integration site of the genetic construct. Alternatively, the haplo-sufficient gene may be located on a different chromosome than the integration site of the genetic construct.

As illustrated in FIG. 11a, the genetic construct may encode i) a first genomic editor that targets the wildtype allele of the haplo-sufficient gene to cause dominant lethality or sterility in both sexes, and ii) a second genomic editor that targets an allele linked to the construct and located upstream from the target site of the first genomic editor to create a premature stop codon which causes recessive sterility or lethality in both sexes.

Preferably, therefore, the first genomic editor creates a dominant negative or a dominant gain of function mutation in a wild-type allele of the target gene. This dominant negative or dominant gain of function mutation causes dominant lethality or sterility in both males and females.

For localised suppression, the first genomic editor preferably creates a dominant lethal or sterile phenotype in female organisms or in both sexes. Accordingly, in a preferred embodiment, the first genomic editor creates a dominant lethal or sterile mutation in a target gene expressed in the female organism, or in a target gene expressed in the male and female organism.

Preferably, the second genomic editor creates a premature stop codon in an allele of the target gene linked to the genetic construct and located upstream from the target site of the first genomic editor. Accordingly, in one preferred embodiment, the second genomic editor creates a premature stop codon in the haplo-sufficient gene. Preferably, the second genomic editor creates a premature stop codon an allele of the haplo-sufficient gene linked to the genetic construct and located upstream from the target site of the first genomic editor.

This premature stop codon causes recessive sterility or lethality in both males and females. The presence of the premature stop codon prevents the downstream dominant negative mutation from being expressed, and so individuals that inherit one copy of the construct and the associated stop codon, and have one wild-type allele, have nearly normal fitness (though they would be non-viable or sterile if homozygous for the construct and the associated stop codon).

Specificity for the allele linked to the construct could be achieved by recoding the allele linked to the construct such that it is still functional but can be targeted by the genome editor, whereas the wildtype allele would be immune. Accordingly, in one embodiment, the allele of the target gene linked to the genetic construct has been recoded such that it can be targeted by the second genomic editor. Preferably, the wildtype allele is not recognised by the second genomic editor.

As illustrated in FIGS. 11b and 11c, the genetic construct may be configured to induce recessive sterile or lethal mutations in a second gene, separate to that which is targeted to induce the dominant sterile or lethal mutations.

Accordingly, in one embodiment, the second genomic editor creates a recessive lethal or sterile mutation in the haplo-sufficient gene. Preferably, the first genomic editor creates a dominant lethal or sterile mutation in a haplo-insufficient gene or any gene in which it is possible to create dominant lethal or sterile mutations in males and females, or in females only. The haplo-sufficient and/or haplo-insufficient gene may be located at any distance from the genetic construct. In one embodiment, the gene that is targeted by the first genomic editor is a haplo-insufficient gene. Alternatively, the gene that is targeted by the first genomic editor may be any gene in which it is possible to create dominant lethal or sterile mutations in males and females, or in females only.

In this embodiment, the construct can either be linked (FIG. 11b) or unlinked (FIG. 11c) to a haplo-insufficient gene, or any gene in which it is possible to create dominant lethal or sterile mutations in males and females, or in females only. Accordingly, in one embodiment, the construct is linked to the gene that is targeted by the first genomic editor (i.e. the haplo-insufficient gene, or any gene in which it is possible to create dominant lethal or sterile mutations in males and females, or in females only). It will be appreciated by the skilled person that linked means that the construct and the haplo-insufficient gene, or any gene in which it is possible to create dominant lethal or sterile mutations in males and females, or in females only, are located on the same chromosome. In one embodiment, the construct and the haplo-insufficient gene, or any gene in which it is possible to create dominant lethal or sterile mutations in males and females, or in females only, are located within 10, 8, 6, 4, 2 or 1 centiMorgans of one another.

Alternatively, in another embodiment, the genetic construct is unlinked to the gene that is targeted by the second genomic editor (i.e. the haplo-insufficient gene, or any gene in which it is possible to create dominant lethal or sterile mutations in males and females, or in females only). It will be appreciated by the skilled person that unlinked means that the construct and the haplo-insufficient gene, or any gene in which it is possible to create dominant lethal or sterile mutations in males and females, or in females only, are located on different chromosomes.

As shown in FIG. 11c, the genetic construct may further comprise a rescue copy of the target gene that is not recognised by the genomic editor. Accordingly, in one embodiment, the genetic construct further comprises a nucleotide sequence encoding a rescue copy of the target gene. The nucleotide sequence encoding the rescue copy of the target gene is not recognised by the genomic editor. For example, in an embodiment in which the genomic editor is a CRISPR/Cas9-based genomic editor, the nucleotide sequence encoding the rescue copy of the target gene differs from the wild-type nucleotide sequence recognised by a gRNA of the genomic editor, such that the rescue copy is not edited by the Caso protein of the genomic editor.

However, the nucleotide sequence encoding the rescue copy of the target gene may also be designed such that it encodes the same amino acid sequence as the wild-type nucleotide sequence, or a functionally equivalent amino acid sequence. In an embodiment in which the genomic editor produces a dominant negative mutation, then preferably the rescue copy on the construct is configured to dilute out the effect of the mutation. Preferably, therefore, the nucleotide sequence encoding the rescue copy of the target gene is configured to have higher expression levels, to achieve this dilution effect. Alternatively, in an embodiment in which the genomic editor produces a knock-out of a haplo-insufficient gene, then preferably the rescue copy comprises a nucleotide sequence encoding the knocked-out protein. In either case, fitness is rescued if there is one edited allele, but not if there are two edited alleles, so that organisms that are heterozygous for the construct and have one edited allele have normal or near normal fitness, whereas organisms that are heterozygous for the construct and have two edited alleles would suffer the consequences of the mutation.

Advantageously, with this aspect of the invention, all the fitness costs imposed by the construct could potentially be suppressed by the use of a Caso inhibitor. This would allow for the transgenic organism to be maintained as a pure breeding line, reducing rearing costs and time.

All three different types of suppression can be achieved by targeting the female-specific exon of homologs of the doublesex gene to make dominant negative mutations, according to the insertion site of the editor (or the location of the associated disruption). Insertion in (or disruption of) the 5′ region needed in both sexes gives localised suppression (FIG. 2a right); insertion in (or disruption of) the female-specific exon gives non-localised complete suppression (FIG. 6a right); and insertion in (or disruption of) the male-specific exon gives non-localised partial suppression (FIG. 10d).

Suitable organisms which may be targeted using the genetic construct of the invention include disease vectors, agricultural pests or undesired invasive species. Preferably, the types of pest species may include, but is not limited to, arthropods, other invertebrates (e.g., mussels), mammals, other vertebrates (e.g., fish) and weeds.

Suitable arthropods which may be targeted using the gene drive genetic construct of the invention include insects, arachnids, myriapods or crustaceans. Preferably, the arthropod is an insect. Preferably, the arthropod, and most preferably the insect, is a disease-carrying vector or pest (e.g. agricultural pest), which can infect, cause harm to, or kill, an animal or plant of agricultural value, for example, Anopheline species, Aedes species (as a disease vector), Ceratitis capitata, or Drosophila species (as an agricultural pest).

Alternatively, the insect may be a mosquito. Preferably, the mosquito is of the subfamily Anophelinae. Preferably, the mosquito is selected from a group consisting of: Anopheles gambiae; Anopheles coluzzi; Anopheles merus; Anopheles melas; Anopheles arabiensis; Anopheles quadriannulatus; Anopheles stephensi; Anopheles arabiensis; Anopheles funestus; Anopheles albimanus; Anopheles darlingi; and Anopheles sinensis. Alternatively, the mosquito may be of the genus Aedes, preferably Aedes aegypti or Aedes albopictus. Alternatively, the mosquito may be in the genus Culex, preferably Culex pipiens or Culex quinquefasciatus.

Alternatively, the insect may be a tephritid fruit fly, preferably Ceratitis capitata (Mediterranean fruit fly), Bactrocera spp., (including B. oleae, B. dorsalis, tryoni), or Anastrepha spp. (including A. grandis, A. ludens, A. obliqua, A. suspensa).

Alternatively, the insect may be a sandfly, preferably a species that transmits disease. Preferably, the sandfly may be a Phlebotomus spp, or a Lutzomyia spp., preferably L. longipalpis.

Alternatively, the insect may be another dipteran, preferably Glossina spp. (tsetse fly), Rhagoletis pomonella (apple maggot), Drosophila suzukii (spotted wing drosophila), Cochliomyia hominivorax (screwworm), or Lucilia cuprina (Australian sheep blowfly).

Alternatively, the insect may be a lepidopteran, preferably a lepidopteran that is an agricultural pest. Preferably, the lepidopteran is selected from one of the following: Pectinophora gossypiella (pink bollworm), Lymantria dispar (gypsy moth), Epiphyas postvittana (light brown apple moth), Lobesia botrana (European grapevine moth), Cydia pomonella (codling moth), Synanthedon myopaeformis (apple clearwing moth), Plutella xylostella (diamondback moth), Spodoptera frugiperda (fall armyworm), Phthorimaea absoluta (formerly Tuta absoluta; tomato pinworm), Chilo partellus (stem borer), Helicoverpa armigera (old world bollworm), and Helicoverpa zea (corn earworm).

Alternatively, the insect may be a coleopteran, preferably a coleopteran that is an agricultural pest. Preferably, the coleopteran is selected from one of the following: Rhynchophorus ferrugineus (red palm weevil), Hypothenemus hampei (coffee berry borer beetle), Sternochetus frigidus (mango pulp weevil), and Sitona obsoletus (clover root weevil).

Alternatively, the insect may be a mealybug, preferably a mealybug that is an agricultural pest Preferably, the mealybug is selected from Phenacoccus manihoti (cassava mealybug), and Phenacoccus solenopsis (mealybug)

Alternatively, the organism may be another arthropod, preferably a harmful invasive arthropod, such as Pacifastacus leniusculus (signal crayfish).

Alternatively, the organism may be a gastropod, preferably a gastropod that is an intermediate host for a parasite of humans or other vertebrates, such as a member of Bulinus spp., Biomphalaria spp., or Oncomelania spp. Alternatively, the gastropod may be a harmful invasive species, preferably Crepidula fornicata (common slipper limpet), or Pomacea canaliculata (golden apple snail).

Alternatively, the organism may be a bivalve, preferably a harmful invasive bivalve, such as Dreissena polymorpha (zebra mussel), Limnoperna fortunei (golden mussel), or Corbicula fluminea (invasive Asian clam).

Alternatively, the organism may be a fish, preferably an invasive fish. Preferably, the fish may be a Cyprinus carpio (common carp), Petromyzon marinus (sea lamprey), Salvelinus fontinalis (brook trout), or Pseudorasbora parva (stone moroko),

Alternatively, the organism may be an amphibian, preferably a harmful invasive amphibian, such as Rhinella marina (cane toad), or Lithobates catesbeianus (American bullfrog).

Alternatively, the organism may be a mammal, preferably a harmful invasive mammal. Preferably, the mammal is Mus musculus (house mouse), Mus domesticus (house mouse), Rattus norvegicus (brown rat), Rattus rattus (black rat), Rattus exulans (Polynesian rat), Oryctolagus cuniculus (common rabbit), Felis silvestris catus (feral cat), Sciurus carolinensis (grey squirrel), or Trichosurus vulpecula (common brushtail possum).

Alternatively, the organism may be a plant, preferably a weed or harmful invasive plant. Preferably, the plant is selected from the group consisting of: Amaranthus palmeri, Amaranthus tuberculatus, Alopecurus myosuroides, Lolium rigidum, Kochia scoparia, Centaurea maculosa, Lantana camara, Ambrosia artemisiifolia, and Eragrostis plana.

In some embodiments, the organism comprises a disrupted allele of the haplo-sufficient gene. Accordingly, in some embodiments, the disruption of the haplo-sufficient gene is caused by means other than the integration of the genetic construct. Preferably, the disruption is caused by the introduction of a knock-out mutation, preferably wherein the knockout mutation is caused by the introduction of a premature stop codon, optionally by insertion of a sequence. Accordingly, in some embodiments, the organism comprises a knock-out mutation of the haplo-sufficient gene, preferably wherein the organism comprises a premature stop codon. Alternatively, the knock-out mutation may be caused by the deletion of part or all of the haplo-sufficient gene. Accordingly, in some embodiments, the organism comprises a deletion of part or all of the haplo-sufficient gene.

As described above, the genomic editor may target the haplo-sufficient gene into which, or near to which, the genetic construct integrates. In particular, for localised suppression, the disrupted gene needs to be haplo-sufficient and essential for viability and reproduction in both sexes. Accordingly, in one embodiment, the target gene is a wild type allele of the disrupted haplo-sufficient gene.

Alternatively, in another embodiment, the target gene may be any other gene where a dominant negative or a dominant gain of function mutation can be produced. Dominant negative mutations include mutations that result in proteins that interfere with the normal function of the wild type allele. Dominant gain of function mutations are those that result in proteins taking on new functions. Accordingly, in one embodiment, the target gene may be a haplo-sufficient gene. The target haplo-sufficient gene may be a different gene to the haplo-sufficient gene that is disrupted by the integration of the genetic construct.

One common way of making a dominant negative mutation is through ‘multimer poisoning’. If, for example, the protein usually works as a dimer, and wildtype and mutant protein molecules are equally common and combine at random, then only ¼ of the dimers will consist of two wildtype molecules and function properly, which may be too few for proper function. Accordingly, in one embodiment, the target gene may encode a protein that acts as a multimer.

Classic examples of classes of genes that can mutate to give dominant negative mutations include homodimeric membrane receptors and transcription factors. Preferably, therefore, the target gene encodes a transcription factor or a membrane-bound protein, preferably a homodimeric membrane receptor.

In an embodiment in which the organism is an insect, the target gene may be doublesex (dsx), or a homolog of the doublesex gene. Knock-out mutations of doublesex are bisex sterile, while stop codons introduced in the female-specific exon can be dominant female sterile. More specifically, the 5′ region of this gene encodes a DNA binding domain that is expressed in both sexes and knockouts in this region give homozygous intersex and sterility in both sexes, with fertility more-or-less normal in heterozygotes. Accordingly, in one embodiment, the genomic editor is inserted into and disrupts the 5′ region of doublesex encoding a DNA binding domain that is expressed in a male and female organism.

Downstream of this region there is a female-specific exon, and premature stop codons in this can give dominant female sterility. Thus, in one preferred embodiment, the genomic editor targets the female-specific exon of the doublesex gene. Preferably, the genomic editor introduces a premature stop codon into the female-specific exon of the doublesex gene. For example, this may be achieved by base editing, by reverse prime editing, by introducing indel mutations, or by stimulating homing of a stop codon that has been introduced by the investigator into the inventive haplotype. This will result in the wild type allele being mutated into a dominant female-specific sterile allele. Dominant female sterile edits on the chromosome containing the construct will not be expressed because of the upstream stop codon caused by insertion of the construct.

Alternatively, any gene that is haplo-sufficient yet editable to give a dominant sterile or lethal phenotype can be used. In one embodiment, the haplo-sufficient gene may be selected from the group consisting of homologs of the following Drosophila genes: 5-HT2A, Antp, BicD, cact, chic, Col4a1, crn, cype, dare, dl, dpp, Fs(2)Ket, gro, hb, hh, hop, Hsc70-3, Hsc70-4, ken, l(1)10Ad, l(1)10Ae, l(2)25Ca, l(2)40Ff, l(2)46Fb, l(2)DTS18SP, l(2)DTS19, l(2)DTS20, l(2)DTS6, l(2)DTS8, l(2)DTS9, l(2)M167, lt, M(3)80, nos, Prosbeta6, puc, rl, Scr, snf, stmA, Sxl, tku, Tl, tor, and wupA.

Of the above genes, 15 have an amorphic or loss-of-function allele being a recessive lethal. Accordingly, in a preferred embodiment, the haplo-sufficient gene may be selected from the group consisting of: Antp, BicD, cact, chic, dpp, gro, hh, hop, puc, rl, Scr, snf, Sxl, tkv, and wupA.

In another embodiment, the target gene is a haplo-insufficient gene. Preferably, in this embodiment, the mutation created by the genomic editor results in a knock-out of the haplo-insufficient gene. This is because for a haplo-insufficient gene, having only one active allele is insufficient for normal function. In one embodiment, the haplo-insufficient gene is a haplolethal gene. Haplolethal genes are a special type of haplo-insufficient gene, in which individuals with only one active copy of the gene die early in development, before reproducing.

For example, Drosophila melanogaster comprise at least 43 genes that are haplolethal or haplosterile. The majority of these encode protein components of cytoplasmic ribosomes (Rp genes) or translation initiation factors (eIF genes) and are associated with the Minute syndrome, a characteristic set of phenotypes including short, thin bristles and slower development. Thus, in one preferred embodiment, the genomic editor targets a homolog of a cytoplasmic ribosomal protein or a translation initiation factor.

In another embodiment, the target gene may be a haplo-insufficient gene including those that encode the muscle components actin (Act88F), myosin (Mhc and Mlc2) and tropomyosin (Tm2), as well as a group of closely linked, muscle-related genes regulated by a haplolethal sequence within an intron of the Troponin I (wupA) gene. This subset of genes may also include Hdl, which may correspond to Troponin T (up). Other subsets of haplo-insufficient genes encode homeodomain proteins (Abd-B, Dll, Scr and Ubx), Notch pathway components (Dl, H and N), Polycomb group repressor proteins (Pc and Pcl), apoptosis regulators (lok and p53), and melanin biosynthetic enzymes (b and e).

For some embodiments, the gene that is disrupted, while essential in nature, is not essential in the lab, in order to facilitate rearing a homozygous pure-breeding line. As such, the gene disruption may be auxotrophic and able to be compensated for by a dietary supplement. Accordingly, in one embodiment, the disrupted gene is involved in the biosynthesis of purine, pyrimidines, fatty acids, or is rescuable by dietary supplement with these factors, or fructose or lineolate. The target gene may be a homolog of the Drosophila genes rudimentary, rudimentary-like, Dhod, ade2, ade3, ade4, ade5, bur, Pgd, or SREBP.

The genetic construct comprises a nucleotide sequence which encode a sequence-specific genomic editor which edits a naturally existing sequence in the genome to create a lethal or sterile mutation. Several genomic editors are well-known to those skilled in the art. The genomic editor may be selected from a group consisting of: a transcription activator-like effector nuclease (TALEN) genomic editor; Zinc finger nuclease (ZFN) genomic editor; and a CRISPR-based genomic editor. Preferably, the genomic editor is a CRISPR-based genomic editor, most preferably a CRISPR-Cpf1-based or CRISPR-Cas9-based genomic editor. However, it will be appreciated that other nucleases used in CRISPR-based genomic engineering methods are known and may be used in accordance with the invention.

Accordingly, in an embodiment in which the genomic editor is a CRISPR-based genomic editor, the genomic editor comprises a first nucleotide sequence that is capable of hybridising to the target gene. Preferably, the first nucleotide sequence which is capable of hybridising to the target gene is a guide RNA (gRNA). In a preferred embodiment, the genomic editor comprises a nucleotide sequence encoding at least one gRNA, preferably two gRNA, or three gRNA. In one preferred embodiment, the genomic editor comprises a nucleotide sequence encoding two gRNAs. Advantageously, more than one gRNA increases editing efficiency and/or prevents the evolution of resistance.

Preferably, the CRISPR-based genomic editor further comprises a second nucleotide sequence encoding a CRISPR nuclease, preferably a Cpf1 or Cas9 nuclease, and most preferably a Cas9 nuclease, or a derivative thereof to allow for DNA nicking, base editing, prime editing, or other types of edit. The sequences of the CRISPR nuclease and encoding nucleotides are known in the art. The first and second nucleotide sequences may be on separate nucleic acid molecules forming two genetic constructs, which act in tandem (i.e. in trans) as the genetic construct of the invention. Preferably, however, the first and second nucleotide sequences are on, or form part of, the same nucleic acid molecule, thereby creating the genetic construct of the invention. Preferably, the second nucleotide sequence encoding the nuclease is disposed 5′ of the first nucleotide sequence encoding a nucleotide sequence that is capable of hybridising to the target gene.

The part of the nucleotide sequence that is capable of hybridising to the target gene (i.e. the guide RNA) is known as a protospacer. In order for the nuclease to function, it also requires a specific protospacer adjacent motif (PAM) that varies depending on the bacterial species of the nuclease encoding gene. The most commonly used Cas9 nuclease recognises a PAM sequence of NGG that is found directly downstream of the target sequence in the genomic DNA on the non-target strand. Recognition of the PAM by the nuclease is believed to destabilise the adjacent sequence, allowing interrogation of the sequence by the guide RNA, and resulting in RNA-DNA pairing when a matching sequence is present. The PAM is not present in the guide RNA sequence, but needs to be immediately downstream of the target site in the genomic DNA.

The skilled person would understand that the nucleotide sequence (i.e. guide RNA) that is capable of hybridising to the target gene may further comprise a CRISPR nuclease binding sequence, preferably a Cpf1 or Cas9 nuclease binding sequence, and most preferably a Cas9 nuclease binding sequence. The CRISPR nuclease binding sequence creates a secondary binding structure which complexes with the nuclease, for example a hairpin loop. The PAM on the host genome is recognised by the nuclease.

The CRISPR-based genomic editor further comprises at least one promoter sequence, which drives expression of the first and second nucleotide sequence. In other words, expression of the first and second nucleotide sequences is under the control of the same promoter. Alternatively, the CRISPR-based genomic editor may comprise at least two promoter sequences, such that expression of the first and second nucleotide sequence is under the control of separate promoters. In one embodiment, therefore, the editor comprises a first promoter sequence operably linked to the first nucleotide sequence and a second promoter sequence operably linked to the second nucleotide sequence. The first and second promoter sequence may be any promoter sequence that is suitable for expression in an organism, and which would be known to those skilled in the art. Accordingly, the guide RNA is preferably expressed under control of the first promoter, and the nuclease is expressed under control of the second promoter.

Preferably, the first promoter is a polymerase III promoter, and most preferably a polymerase III promoter which does not add a 5′cap or a 3′polyA tail. More preferably, the promoter is a U6 promoter.

Preferably, the genomic editor comprises control sequences that ensure the editor is active in a male and/or female germline, such that a large fraction of the progeny inherit the lethal or sterile mutation. Accordingly, in one embodiment, the genetic construct further comprises a nucleotide sequence comprising a control sequence that ensures the genomic editor is active in a male and/or female germline. More preferably, the genetic construct comprises a first and second nucleotide sequence comprising a first and second control sequence. Preferably, the first and second nucleotide sequences comprising the first and second control sequences flank the genomic editor. Preferably, the control sequences include but are not limited to promoters, enhancers, terminators, or other regulatory sequences. Alternatively, the genomic editor may be active in other tissues apart from the germline.

Control sequences specifying gene expression in the germline can be taken from native genes in the target organism that show germline expression. Preferably, control sequences include homologs of the Drosophila genes vasa, nanos, zpg, exu, mei-W68 (spo11), and betaTubulin. Alternatively, such genes can be found by RNAseq experiments on the target organism comparing gene expression in germline and somatic tissue. For mice, rats, and other mammalian target species, useful sequences are referred to by Lai et al. (2022: https://www.biorxiv.org/content/10.1101/2022.08.30.505951V1).

In one embodiment, the genetic construct comprises a nucleotide sequence encoding a fluorescent marker. For example, the nucleotide sequence may encode green fluorescent protein (GFP). Advantageously, the fluorescent marker allows tracking of the presence of the construct.

Preferably, when transcribed, the first nucleotide sequence, which encodes a nucleotide sequence (i.e. the guide RNA) which hybridises to the target gene, targets the editor to the target gene.

Preferably, the genetic construct is inserted into the genome by homologous recombination or homology-directed repair. Accordingly, preferably, the nucleotide sequence configured to disrupt the haplo-sufficient gene, comprises a nucleotide sequence capable of hybridising to the haplo-sufficient gene. Preferably, the nucleotide sequence configured to disrupt the haplo-sufficient gene is substantially complementary or homologous to at least a region of the haplo-sufficient gene such that homologous recombination occurs therebetween. Preferably, the genetic construct comprises nucleotide sequences which flank the nucleotide sequence encoding a genomic editor, wherein each flanking sequence is substantially complementary or homologous to at least a region of the haplo-sufficient gene such that homologous recombination occurs therebetween.

Preferably, the or each nucleotide sequence configured to disrupt the haplo-sufficient gene is substantially complementary or homologous to the nucleotide sequence of the haplo-sufficient gene, such that the genetic construct is integrated into the genome.

In another embodiment, the genetic construct is inserted into the genome via recombinase-mediated cassette exchange, a technique which would be known to those skilled in the art. Accordingly, preferably, the genetic construct further comprises integrase attachment sites (preferably attB integrase attachment sites), which, respectively, flank the nucleotide sequence encoding the genomic editor.

In one preferred embodiment, the genetic construct is introduced into the genome comprising a docking construct, wherein the docking construct comprises integrase attachment sites, preferably attP integrase attachment sites, that are flanked by 5′ and 3′ homology arms that are homologous to the genomic sequences flanking the insertion/integration site (whether inside or near to the haplo-sufficient gene), such that the docking construct is introduced into the genome by homology directed repair. The genetic construct is preferably inserted into the genome via recombinase-mediated cassette exchange, wherein the docking construct is exchanged for the genetic construct through the action of an integrase, preferably φC31 integrase.

The genetic construct may for example be a plasmid, cosmid or phage and/or be a viral vector. Such recombinant vectors are highly useful in the delivery systems of the invention for transforming cells. The nucleic acid sequence may preferably be a DNA sequence. The genetic construct may further comprise a variety of other functional elements including a suitable regulatory sequence for controlling expression of the genetic construct upon introduction of the construct in a host cell. The construct may further comprise a regulator or enhancer to control expression of the elements of the constructs required. Tissue specific enhancer elements, for example promoter sequences, may be used to further regulate expression of the construct in cells of an organism.

In a third aspect, there is provided the use of the genetic construct of the first aspect, to disrupt a haplo-sufficient gene needed for survival or reproduction in a male and/or female organism, or to integrate into or near to a disrupted allele of the haplo-sufficient gene, such that a homozygote for the genetic construct is lethal or sterile, and to create a dominant lethal or sterile mutation in a target gene expressed in a male and/or female organism, such that a heterozygote for the dominant lethal or sterile mutation is lethal or sterile, and/or male and/or female organism comprising the mutation is unable to reproduce.

In some embodiments, the disrupted allele of the haplo-sufficient gene comprises a knock-out mutation, optionally wherein the knockout mutation is caused by the introduction of a premature stop codon. Alternatively, the knock-out mutation may be caused by the deletion of part or all of the haplo-sufficient gene.

In some embodiments, there is provided the use of the genetic construct of the first aspect, to disrupt a haplo-sufficient gene needed for survival or reproduction in a male and/or female organism, such that a homozygote for the genetic construct is lethal or sterile, and to create a dominant lethal or sterile mutation in a target gene expressed in a male and/or female organism, such that a heterozygote for the dominant lethal or sterile mutation is lethal or sterile and/or a male and/or female organism comprising the mutation is unable to reproduce.

In a fourth aspect, there is provided the use of the genetic construct of the second aspect, to integrate into a location outside of a haplo-sufficient gene needed for survival or reproduction in a male and/or female organism, and to create a dominant lethal or sterile mutation in a target gene expressed in the male and/or female organism, such that a heterozygote for the dominant lethal or sterile mutation is lethal or sterile, and to create a recessive lethal or sterile mutation in the haplo-sufficient gene expressed in the male and/or female organism, such that a homozygote for the recessive lethal or sterile mutation is lethal or sterile.

In a fifth aspect, there is provided a method of producing a genetically modified organism, the method comprising introducing, into an organism, the genetic construct according to the first or second aspect.

In a sixth aspect, there is provided a method of producing a genetically modified organism, the method comprising introducing, into an organism, a genetic construct comprising a first nucleotide sequence configured to disrupt a haplo-sufficient gene needed for survival or reproduction in a male and/or female organism, or to integrate into or near to a disrupted allele of the haplo-sufficient gene, and a second nucleotide sequence encoding a genomic editor that creates a dominant lethal or sterile mutation in a target gene of the male and/or female organism, such that a homozygote for the genetic construct is lethal or sterile and/or a heterozygote for the dominant lethal or sterile mutation is lethal or sterile.

In some embodiments, the disrupted allele of the haplo-sufficient gene comprises a knock-out mutation, optionally wherein the knockout mutation is caused by the introduction of a premature stop codon. Alternatively, the knock-out mutation may be caused by the deletion of part or all of the haplo-sufficient gene.

In some embodiments, there is provided a method of producing a genetically modified organism, the method comprising introducing, into an organism, a genetic construct comprising a first nucleotide sequence configured to disrupt a haplo-sufficient gene needed for survival or reproduction in a male and/or female organism, and a second nucleotide sequence encoding a genomic editor that creates a dominant lethal or sterile mutation in a target gene of the male and/or female organism, such that a homozygote for the genetic construct is lethal or sterile and/or a heterozygote for the dominant lethal or sterile mutation is lethal or sterile.

In a seventh aspect, there is provided a method of producing a genetically modified organism, the method comprising introducing, into an organism, a genetic construct comprising a first nucleotide sequence configured to integrate into a location outside of a haplo-sufficient gene needed for survival or reproduction in a male and/or female organism, and a second nucleotide sequence encoding: (i) a first genomic editor that creates a dominant lethal or sterile mutation in a target gene expressed in the male and/or female organism; and (ii) a second genomic editor that creates a recessive lethal or sterile mutation in the haplo-sufficient gene, such that a homozygote for the recessive lethal or sterile mutation is lethal or sterile.

The genome editing method or technique may be carried out in vivo, in vitro or ex vivo. Preferably, the haplo-sufficient gene, the target gene, the genetic construct, and the organism of the third to seventh aspects are as defined in the first or second aspect.

The genetic construct may be introduced directly into an organism host cell, preferably an organism host cell present in an organism embryo, by suitable means, e.g. direct endocytotic uptake. The construct may be introduced directly into cells of a host organism (e.g. a mosquito) by transfection, infection, electroporation, microinjection, cell fusion, protoplast fusion or ballistic bombardment. Alternatively, constructs of the invention may be introduced directly into a host cell using a particle gun.

The inventors also surprisingly discovered that a second construct may be used to increase the frequency of the first genetic construct in a population. Accordingly, the method according to the fifth, sixth or seventh aspect may further comprise introducing into the organism a second genetic construct comprising a nucleotide sequence configured to increase the frequency of the first genetic construct. In this embodiment, the first genetic construct is the genetic construct that disrupts, or is associated with a disruption of, the haplo-sufficient gene.

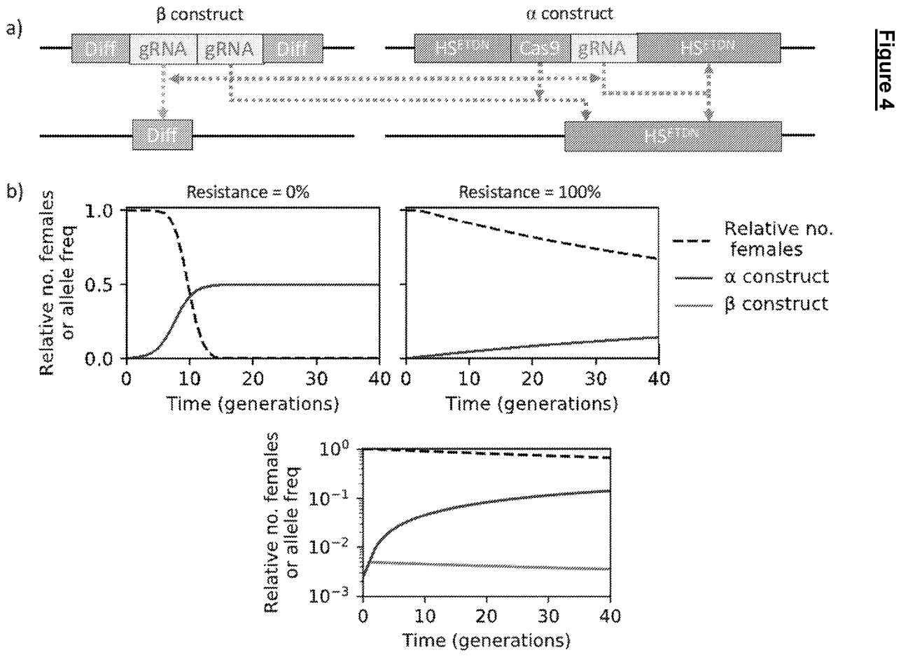

For example, the second construct may comprise a nucleotide sequence encoding a gRNA that, in the presence of Cas9 nuclease encoded by the first construct, cleaves the wild type allele of the disrupted gene and thereby increases the transmission of the construct to the next generation, either by homing or by disrupting the transmission of the chromosome with the wild type allele. Alternatively, the second construct may also encode Cas9 or any other appropriate nuclease. In either case, the second construct gives the first construct a temporary boost in frequency, making it even more efficient while still staying localised (FIG. 3). Accordingly, in one embodiment, the second genetic construct comprises a nucleotide sequence encoding a guide RNA that targets the integration site of the first genetic construct. As such, the second construct facilitates a temporary increase in the frequency of the first genetic construct.

Alternatively, the second construct may comprise a first and second nucleotide sequence encoding a first and second gRNA, respectively. The first gRNA preferably allows the first construct to home, and the second gRNA preferably allows the second construct to home in the presence of the first construct, creating a double drive which may spread to additional populations, and whose spread may be controlled by exploiting pre-existing sequence differences between target and non-target populations (FIG. 4). Accordingly, in another embodiment, the second genetic construct preferably comprises a first nucleotide sequence encoding a first guide RNA that targets the integration site of the first genetic construct, and a second nucleotide sequence encoding a second guide RNA that targets the integration site of the second genetic construct. As such, this facilitates homing and increases the frequency of both the first and second genetic constructs when they are inherited together.

Alternatively, other methods of boosting the first genetic construct may be used. For example, in one embodiment, the first genetic construct may comprise a nucleotide sequence encoding a rescue construct that masks the effect of mutations produced by the second construct. Alternatively, further genetic constructs may be used which cause the second construct to increase in frequency.

In an eighth aspect, there is provided a genetically modified organism obtained or obtainable by the method of the fifth, sixth or seventh aspect.

In a ninth aspect, there is provided a genetically modified organism comprising a disrupted haplo-sufficient gene needed for survival or reproduction in a male and/or female organism, and a nucleotide sequence encoding a genomic editor that creates a dominant lethal or sterile mutation in a target gene, such that the male and/or female organism comprising the mutation is unable to reproduce.

Preferably, the haplo-sufficient gene has been disrupted by a genetic construct as defined in the first aspect. Alternatively, the genetic construct has been inserted into or near to a disrupted allele of the haplo-sufficient gene. Preferably, the haplo-sufficient gene, the target gene, the genetic construct, and the organism is as defined in the first aspect.

In a tenth aspect, there is provided a genetically modified organism comprising a disrupted haplo-sufficient gene needed for survival or reproduction in a male and/or female organism, and a nucleotide sequence encoding a first genomic editor that creates a dominant lethal or sterile mutation in a target gene expressed in the male and/or female organism, such that a heterozygote for the dominant lethal or sterile mutation is lethal or sterile, and a second genomic editor that creates a recessive lethal or sterile mutation in the haplo-sufficient gene expressed in the male and/or female organism, such that a homozygote for the recessive lethal or sterile mutation is lethal or sterile.

In an eleventh aspect, there is provided a method of suppressing a wild type population of an organism, the method comprising breeding a genetically modified organism comprising a genetic construct comprising a first nucleotide sequence configured to disrupt a haplo-sufficient gene needed for survival or reproduction in a male and/or female organism, or to integrate into or near to a disrupted allele of the haplo-sufficient gene, and a second nucleotide sequence encoding a genomic editor that creates a dominant lethal or sterile mutation in a target gene of the male and/or female organism, such that a homozygote for the genetic construct is lethal or sterile and/or a heterozygote for the dominant lethal or sterile mutation is lethal or sterile.

In some embodiments, the disrupted allele of the haplo-sufficient gene comprises a knock-out mutation, optionally wherein the knockout mutation is caused by the introduction of a premature stop codon. Alternatively, the knock-out mutation may be caused by the deletion of part or all of the haplo-sufficient gene.

In some embodiments, there is provided a method of suppressing a wild type population of an organism, the method comprising breeding a genetically modified organism comprising a genetic construct comprising a first nucleotide sequence configured to disrupt a haplo-sufficient gene needed for survival or reproduction in a male and/or female organism, and a second nucleotide sequence encoding a genomic editor that creates a dominant lethal or sterile mutation in a target gene of the male and/or female organism, such that a homozygote for the genetic construct is lethal or sterile.

In a twelfth aspect, there is provided a method of suppressing a wild type population of an organism, the method comprising breeding a genetically modified organism comprising a genetic construct comprising a first nucleotide sequence configured to integrate into a location outside of a haplo-sufficient gene needed for survival or reproduction in a male and/or female organism, and a second nucleotide sequence encoding: (i) a first genomic editor that creates a dominant lethal or sterile mutation in a target gene expressed in the male and/or female organism; and (ii) a second genomic editor that creates a recessive lethal or sterile mutation in the haplo-sufficient gene expressed in the male and/or female organism, such that a homozygote for the recessive lethal or sterile mutation is lethal or sterile.

In a thirteenth aspect, there is provided the use of a genetic construct according to the first or second aspect, to suppress a wild-type population of an organism.

The modified organisms may be released in a numerous different ways in order to suppress the population. For example, in one embodiment, the modified organisms comprising the genetic construct are released into the population. Alternatively, in another embodiment, females that have been mated to males comprising the genetic construct are released into the population. Alternatively, the genetic construct may be contained with pollen that is subsequently released into the population. In other words, an organism at any stage of the life cycle that is appropriate may be released to suppress the wild-type population.

In one embodiment, the suppression may be localised suppression. This means that the construct and suppressive effect will not spread indefinitely throughout a species range, wherever there is gene flow, but rather will be limited to the region around the site(s) of release. This region will typically encompass an area within 2, 4, 6, 8, or 10 times the average dispersal distance of the target species from the release site(s).

In another embodiment, the suppression may be non-localised partial suppression. This means that the construct and suppressive effect will spread out from the release site(s) over successive generations to all regions in the species range with which there is significant gene flow, potentially even after just a single release. The impact in this case is to suppress the target population by at least 20%, 30%, or 40%, and preferably by no more than 70%, 80%, or 90%.

In yet another embodiment, the suppression may be non-localised complete suppression. This means again that the construct and suppressive effect will spread out from the release site(s) over successive generations to all regions in the species range with which there is significant gene flow, potentially even after just a single release. The impact in this case is to suppress the target population by at least 90%, 95%, or 99%, or preferably to eliminate it (notwithstanding that the location may be re-colonised by the target species after elimination).

Preferably, the haplo-sufficient gene, the target gene, the genetic construct, and the organism is as defined in the first or second aspect.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

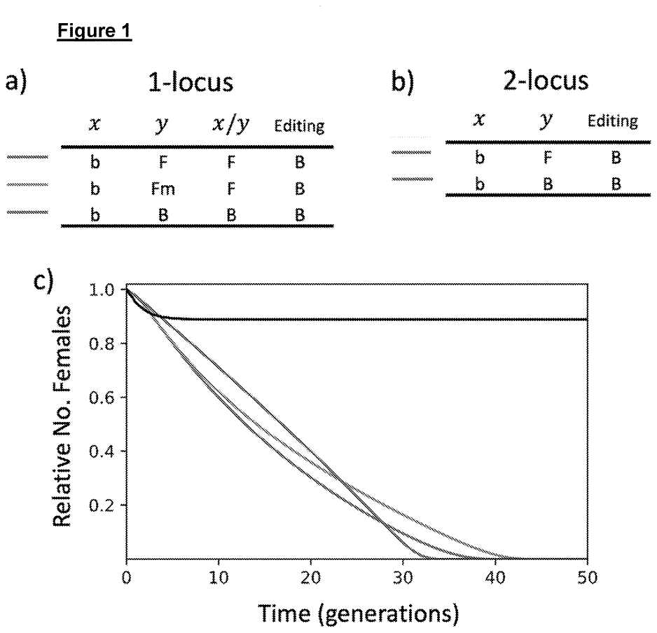

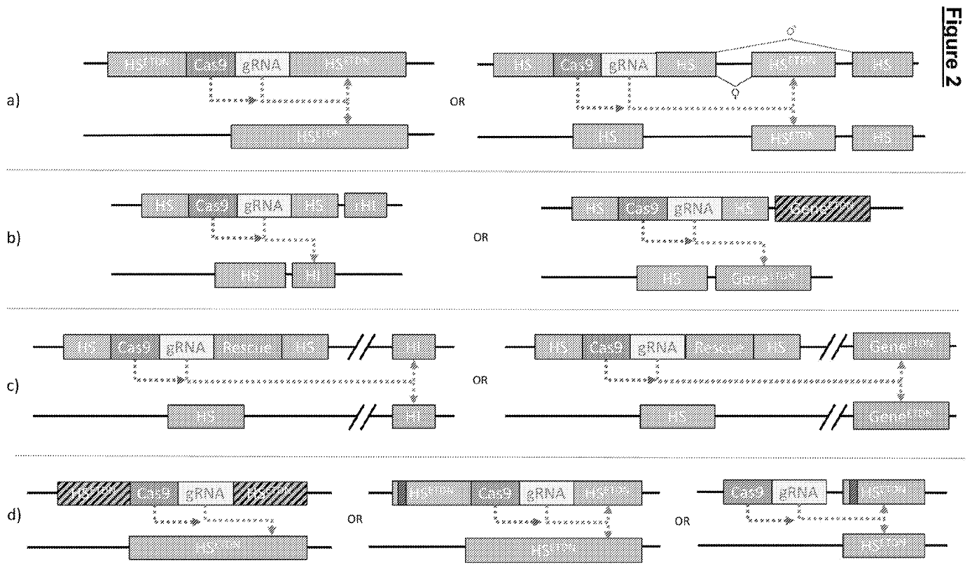

FIG. 1 shows combinations of fitness parameters suitable for localised population suppression. The proposed construct is inserted into and disrupts an essential gene, thereby causing lethality or sterility. The construct encodes a genomic editor that targets a site in the wild type allele to cause a mutation that causes dominant lethality or sterility. The table shows combinations of the fitness effects of gene disruption (x), target site editing (y) and of being heterozygous for the disruption and the edited allele (x/y), and editing expression which lead to efficient localised population suppression after repeat releases in male heterozygotes at 5% of the initial male population. The phenotypic effects of construct insertion or the edit can be male-specific (m or M), female-specific (f or F) or bi-sex (b or B) where lower- and upper-case indicates recessive and dominant effects respectively, and two letters indicate differing dominance patterns in the two sexes. For the single locus design, the x/y column describes the phenotype of individuals that are heterozygous with one copy of the construct and an edited allele on the homologous chromosome. Editing can be controlled by, for example, expressing Cas9 or the gRNA from specific promoters leading to editing in both sexes (B) or only males (M) or females (F). In the localisable strategies shown, the construct insertion and gene disruption causes bi-sex recessive lethality or sterility, and the genome editor may create dominant bisex (green) or dominant female-specific (blue) lethal or sterile edits, or a female-specific dominant lethal or sterile edit with male-specific recessive lethality (orange). Plot shows time course simulations of the relative female population size following repeat releases of the constructs according to the invention, compared to release of sterile males (black). For the purposes of the simulations, all fitness and editing parameters are idealised (i.e., either 0 or 1).

FIG. 2 illustrates proposed molecular configurations for various embodiments of the construct according to the invention, giving localised population suppression. In (a) the construct (shown here for illustration as Cas9 and gRNA) is inserted, by homologous recombination, into a haplo-sufficient essential gene, in which it is possible to induce a dominant negative edit (HS^{ETDN} stands for haplo-sufficient editable to dominant negative). The presence of the construct disrupts the HS^{ETDN} gene causing recessive lethality or sterility (e.g., by introducing a premature stop codon). The construct encodes a genomic editor (e.g. Cas9 and gRNA, though other genome editing tools could be used such as Cas9 derivatives, TALENs, or zinc finger nucleases, etc) that targets a site in the wild type allele of the same gene downstream of the insertion site, to cause a dominant negative edit that, in the absence of the insertion on that chromosome, causes dominant lethality or sterility. The presence of the construct prevents the dominant negative from being expressed, and so individuals that inherit one copy of the construct and one wild type allele have nearly normal fitness (though they would be non-viable or sterile if homozygous for the construct). If the editor acts by creating a double strand break, it should not lead to homing of the construct, as then the suppression would not be localised. The insertion site for the construct is shown as being upstream of the target site, but alternatively it could be downstream, in which case the target site on the chromosome containing the insert would have to be recoded or removed, so that the chromosome with the construct is not recognised by the editor. More generally, the chromosome containing the construct may be deleted for some or all of the wild type HS^{ETDN} gene. The genomic editor could be designed to create a dominant negative mutation which affects both males and females (left), or to target and edit a sequence in a female-specific exon to produce a dominant negative mutation which only affects females (right). This latter approach could be achieved by using a gene such as doublesex. In (b) the construct is again inserted into and disrupts a haplo-sufficient (HS) essential gene, and targets a very closely linked gene to cause a dominant lethal or sterile mutation. The target gene may be haplo-insufficient (HI), and the edit a knock-out, or it may be any other gene where a dominant lethal or sterile mutation can be produced. The dominant mutation created by the editor may be lethal or sterile in both males and females, or only in females. The copy of the gene on the same chromosome as the construct has been modified to be resistant to the editor by changing the target sequence so it is still functional but is no longer recognised by the genome editor, as shown in the left-hand side of the Figure (rHI stands for recoded haplo-insufficient). However, if the editor creates dominant negative mutations (Gene^{ETDN}), then the resistant version may simply be a deletion of the target site or even the entire gene, as shown in the right-hand side of the Figure. In (c), the construct targets a gene editable to a dominant negative or a haplo-insufficient (HI) gene that may be more distantly linked to the construct, including on a different chromosome, and the construct also contains within it a recoded rescue copy of the targeted gene that is not recognised by the editor. In the case of the target gene being HI, the rescue gene would be a modified version of the target gene which is still functional but not recognised by the genome editor. If the edit produces a dominant negative mutation then the rescue would again be a modified version of the target gene which is still functional but not recognised by the genome editor, and may be designed to have higher expression levels, to more effectively dilute out the dominant negative protein. Again, the dominant mutation created by the editor may be lethal or sterile in both males and females, or only in females. For illustration, the genetic constructs in Figures a-c have been depicted as being inserted into and disrupting the haplo-sufficient gene. However, the genetic construct may also be configured to integrate into a disrupted allele of the haplo-sufficient gene, where the disruption may be due to the introduction of a premature stop codon or a deletion or all or part of the gene (d; left and middle). Alternatively the genetic construct may integrate into a location outside of, but near to, a disrupted allele (d; right).

FIG. 3 shows (a) a proposed molecular configuration in which embodiments from FIG. 1 are paired with a second construct containing a gRNA—inserted into a neutral (Ntrl) locus—targeting the insertion locus of the first construct, facilitating a temporary increase in its frequency through homing. The design bBBB (from FIG. 1) implemented at a single locus (shown in FIG. 2a left) is shown for illustration, with the gRNA from the second construct combining with the Caso from the first construct to cleave the wild type allele at the insertion site of the first construct. Alternatively, the second construct may encode its own Cas9 or other RNA-guided nuclease to allow homing of the first construct. (b) Time course simulations showing the allele frequency of the proposed constructs (solid lines) and the impact on relative population size (dashed lines) when there is a single release of both constructs in the same male at 30% of the initial male population size. For the purposes of the simulations, all fitness and cleavage parameters are idealised (0 or 1) and the constructs are assumed to be unlinked. Note that a booster gRNA can be used with any of the molecular configurations shown in FIG. 2 and will have an effect similar to increasing the numbers released.

FIG. 4 shows (a) an example double drive molecular configuration using construct bBBB from FIG. 1. To illustrate, the molecular configuration of the construct is design FIG. 2a left, but the strategy can work with any of the molecular configurations shown in FIG. 2. Two constructs are shown, i.e. an α construct and a β construct. The β construct contains two gRNAs, one targeting the insertion site of the α construct and one targeting its own insertion site, both of which facilitate homing using the Cas9 encoded by the α construct, resulting in both constructs being able to spread from rare. If the β construct is designed to be inserted into a differentiated site, where there is some level of resistance to the gRNA, localisation can be achieved as long as the frequency of resistance is sufficiently low in the target populations, allowing both constructs to spread, and sufficiently high in the non-target population, preventing the β construct from spreading and resulting in dynamics more similar to the design in FIG. 3. (b) Time course simulations showing the allele frequency of the proposed constructs (solid lines) and the impact on relative population size (dashed lines) when released once in the same male at 1% of the initial male population size into populations with 0% and 100% resistance at the differentiated insertion site of the B construct. The bottom plot shows the 100% resistance simulations on a log scale, to show the dynamics of the second construct more clearly. For populations with 0% resistance the allele frequency of both constructs is the same. For the purposes of the simulations, all parameters fitness and cleavage parameters are idealised (o or 1) and constructs are assumed to be unlinked.

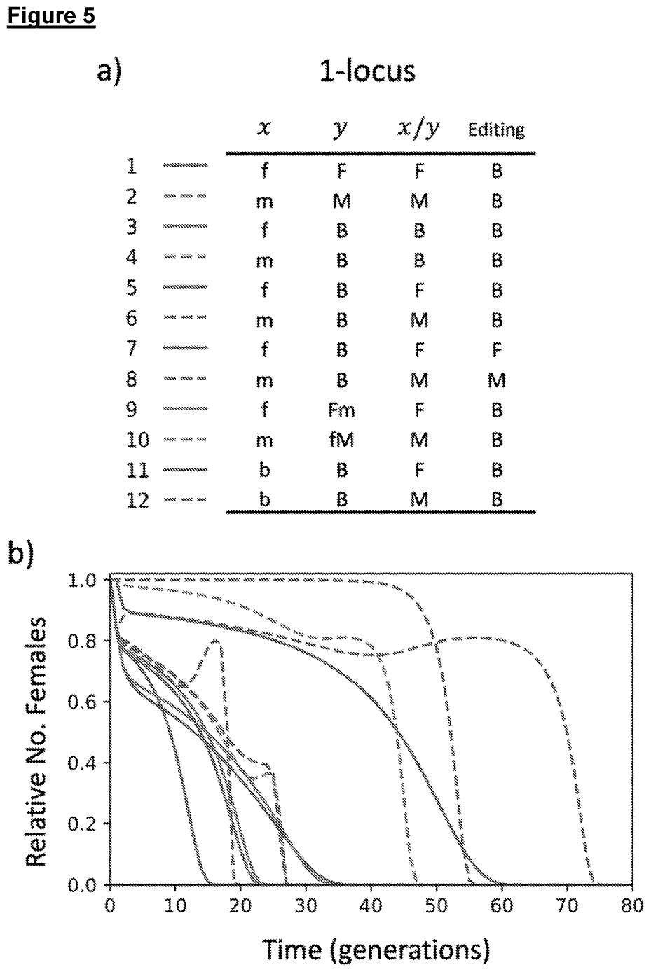

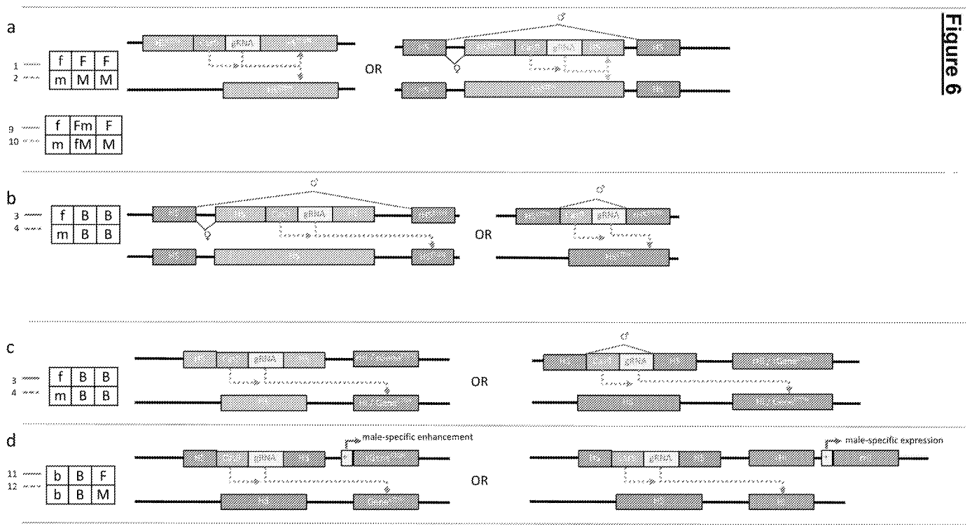

FIG. 5 shows a table showing combinations of the fitness effects of gene disruption (x), target site editing (y) and of being heterozygous for the disruption and the edited allele (x/y), and editing expression which lead to self-sustaining population suppression strategies capable of suppressing a population by more than 99% after a single release of a construct with a single locus molecular design or one with two closely linked loci. The phenotypic effects of construct insertion or the dominant negative edit can be male-specific (m or M), female-specific (f or F) or bi-sex (b or B), where lower- and upper-case indicates recessive and dominant effects respectively, and two letters indicate differing dominance patterns in the two sexes. The x/y column describes the phenotype of individuals that are heterozygous with one copy of the construct and an edited allele on the homologous chromosome. Editing can be controlled by, for example, expressing Cas9 or the gRNA from specific promoters leading to editing in both sexes (B) or only males (M) or females (F). (b) Time course simulations of the relative female population size following a single release of the designs described in in the table when released in heterozygous males at 50% of the initial male population size. For the purposes of the simulations, all fitness and editing parameters are idealised (0 or 1).

FIG. 6 shows example single locus (a, b) and 2-linked loci (c, d) molecular configurations giving complete suppression in a closed random mating population. In each case, if the words “female” and “male” are interchanged, then the strategy will typically still work, though with somewhat different dynamics. Configurations with two linked loci have a haplo-sufficient gene (into which the construct is inserted) closely linked to a haplo-insufficient (HI) gene or a haplo-sufficient gene that is editable to a dominant negative (HS^{ETDN}), and the modification involves both insertion of the construct and recoding of the target site so it is functional but not recognised by the editor. (a) Left: The construct is inserted into and disrupts a haplo-sufficient gene needed only in females, and the edit(s) it produces are female-specific dominant negatives or dominant gain of function mutations. Right: Alternatively, the construct is inserted into and disrupts a female-specific exon of a gene needed in both sexes and the edit(s) it produces are female-specific dominant negatives. In both cases, the insertion site for the construct is shown as being upstream of the site targeted by the editor, but the approach can also work if the insertion site for the editor is downstream of the site targeted by the editor, though in this case the target site on the chromosome containing the construct will need to be modified or removed so that the chromosome containing the construct is not recognised by the editor. (b) The construct is inserted into a haplo-sufficient gene needed in both males and females, but is either inserted into a female-specific exon (left) or has at its ends sequences that ensure it will be spliced out in males (right), with the result that gene function is predominantly disrupted in females. The edit(s) it produces are bisex dominant negatives, and the chromosome that the construct is on is protected from the edits because the target site has been recoded. (c) The construct is inserted into and disrupts a haplo-sufficient gene needed in females (left) or is inserted into and disrupts a haplo-sufficient gene needed in both sexes and has at its ends sequences that ensure it will be spliced out in males (right), with the result that gene function is predominantly disrupted in females. The genomic editor acts on the wild type allele located in a gene different from and tightly linked to the disrupted gene to produce a dominant negative mutation, a dominant gain of function mutation, or a knock-out of a haplo-insufficient gene. In both cases the chromosome that the construct is on is protected from the edits because the target site has been recoded. (d) as in (c) where instead the construct disrupts a haplo-sufficient gene needed in both males and females and the editor targets a closely linked gene needed in both sexes. Left: the editor creates a dominant negative mutation, and the target gene on the chromosome containing the construct has been modified so that it is not recognised by the editor, yet remains functional. Moreover, control sequences have been modified to increase expression specifically in males, to dilute out the effect of the dominant negative edit in males that are heterozygous for the construct and the edit, with the result that only female heterozygotes are lethal or sterile. Right: the editor creates a knock-out mutation in a haplo-insufficient gene, and the target gene on the chromosome containing the construct has been modified so that it is not recognised by the editor, yet remains functional. Moreover, a second copy of the gene, also recoded, so as not to be recognised by the editor, has been inserted with control sequences specifying male-specific expression, with the result that organisms that are heterozygous for the construct and the edit have normal or near normal fitness if they are male, but are sterile or lethal if female. Male-specific expression may be achieved by modifying promoter or enhancer sequences, or by use of an intron that is spliced out only in males, and introduces a premature stop codon if not spliced out.

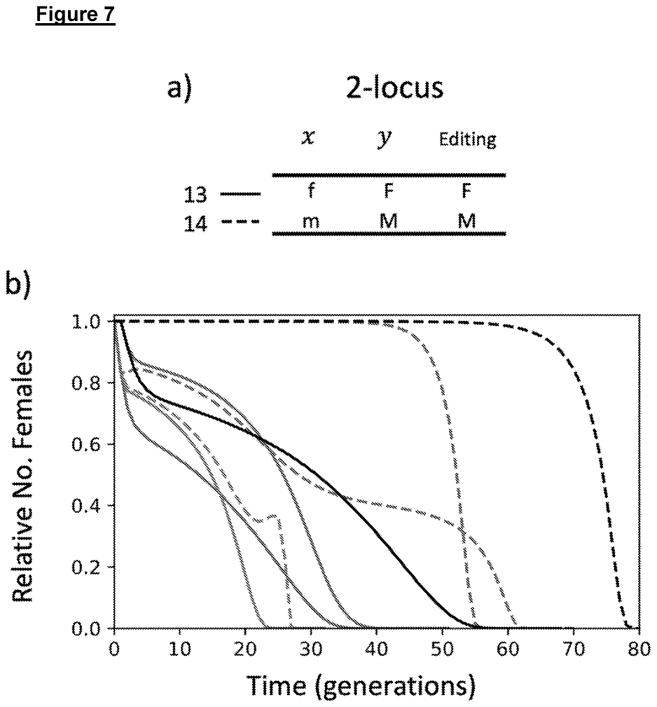

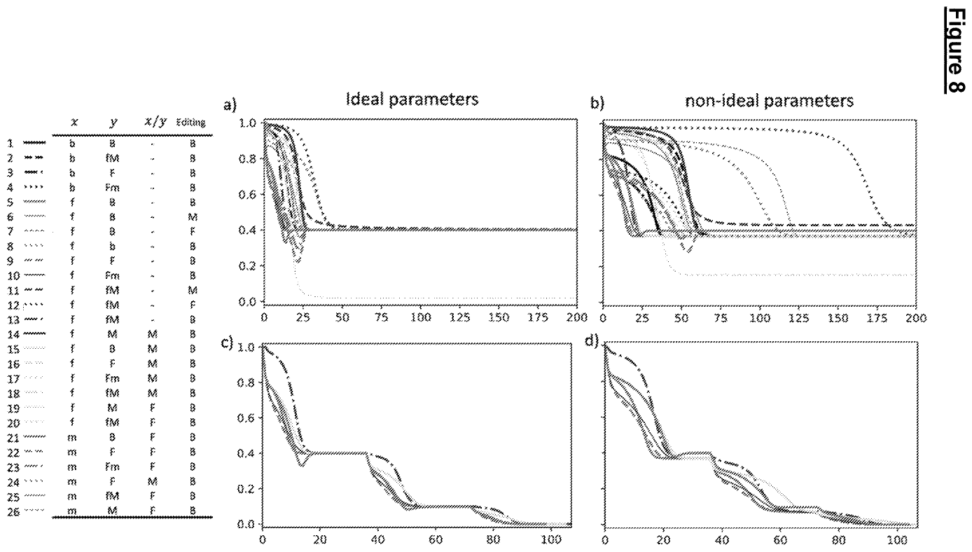

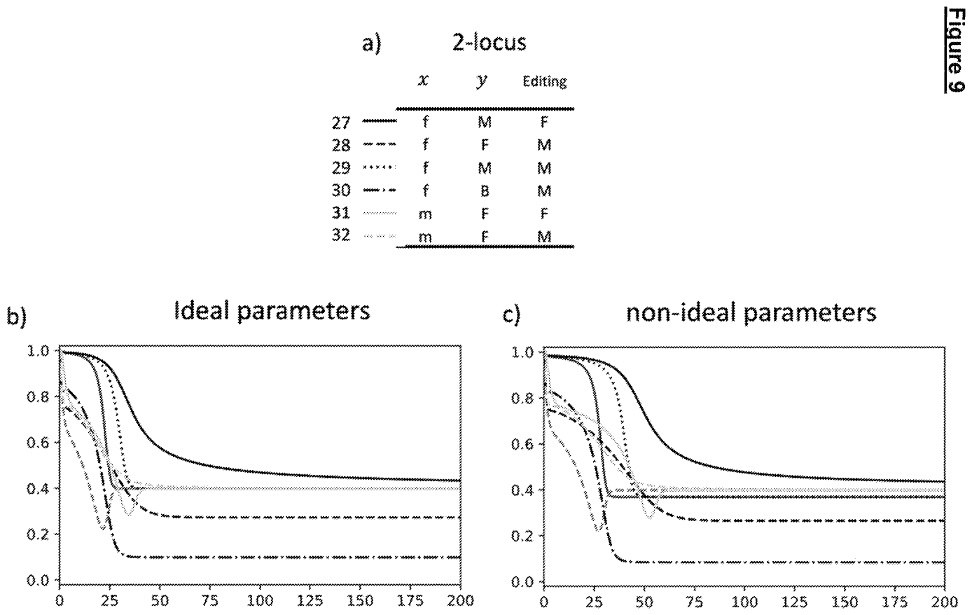

FIG. 7 shows (a) a table showing combinations of the fitness effects of gene disruption (x) and target site editing (y) and editing expression which lead to self-sustaining population suppression strategies capable of suppressing a population by more than 99% after a single release of a construct with a two-locus molecular design. (b) Time course simulations of the relative female population size following a single release of the designs described in the table when released in heterozygous males at 50% of the initial male population size. For the purposes of the simulations, all fitness and editing parameters are idealised (o or 1) and the two loci are assumed to be unlinked. Labels are as in FIG. 5.

FIG. 8 comprises a table showing combinations of the fitness effects of gene disruption (x), target site editing (y) and of being heterozygous for the disruption and the edited allele (x/y), and editing expression which, in our simulations, lead to self-sustaining population suppression strategies capable of partially suppressing a population by between 50% and 99% after a single release of a construct with a single-locus molecular design, or one with two closely linked loci. All labels in the table are as in FIG. 5. The plots show time course simulations of the relative female population size following a single release of the designs described in the table when released in heterozygous males at 50% of the initial male population size with (a) ideal parameters or (b) non-ideal parameters where recessive heterozygotes experience a 5% fitness cost and editing is 80% efficient. Black lines are constructs with a bi-sex recessive phenotype, shades of pink show constructs with a female-specific recessive phenotype and blue are those with a male-specific recessive phenotype. (c) and (d) show time course simulations for the most efficient six strategies (measured by time to reach equilibrium) when an additional two releases are made including one or more constructs of the same design located elsewhere in the genome. In the second release at generation 35, males carry one copy of the first construct and one copy of a second construct. In the third release at generation 70, males contain one copy of each of the three constructs. (c) shows results for idealised parameter values, (d) for non-ideal.

FIG. 9 comprises (a) a Table showing combinations of the fitness effects of gene disruption (x) and target site editing (y) and editing expression which, in our simulations, lead to self-sustaining population suppression strategies capable of partially suppressing a population by between 50% and 99% after a single release of a construct with a two-locus molecular design. Labels are as in FIG. 5. The plots (b and c) show time course simulations of the relative female population size following a single release of the designs described in the table when released in heterozygous males at 50% of the initial male population size with (b) ideal parameters or (c) non-ideal parameters where recessive heterozygotes experience a 5% fitness costs and editing is 80% efficient. In each case, the two loci are unlinked.

FIG. 10 shows example single locus (a-d), 2 linked loci (e-g) and 2 unlinked loci (h, i) molecular configurations giving partial suppression in a closed random mating population. (a) The construct is inserted into and disrupts a haplo-sufficient gene needed in both males and females, and the edit(s) it produces are bisex dominant negatives. In organisms that are heterozygous for the genomic editor and a wild type allele of the HS gene, the editor acts on a wild type target sequence on the chromosome without the construct to produce a dominant negative mutation that is lethal or sterile for both males and females; the target site on the chromosome with the construct has been recoded such that it is still functional but not recognised by the editor (rHS^{ETDN}; no orange arrow). The construct also functions to nullify the effect of the mutation at the RNA level, e.g. by RNA interference to degrade the RNA transcribed from the mutated target gene or RNA editing to revert the RNA transcribed from the mutated target gene, back to the wild type sequence, providing dominant protection against the mutation. Note that if the construct encodes an RNA editing module it is not necessary for the target site on the chromosome with the construct to be recoded since the RNA transcribed from it will also be reverted back to the wild type. (b) Left: The construct is inserted into and disrupts a haplo-sufficient gene needed in females and the edit(s) it produces are female-specific dominant negatives. Right: Alternatively, the construct is inserted into and disrupts a female-specific exon needed in both sexes and the edit(s) it produces are female-specific dominant negatives. (c) The construct is inserted into a haplo-sufficient gene needed in both males and females, but is either inserted into a female-specific exon (left) or has at its ends sequences that ensure it will be spliced out in males (right), with the result that gene function is predominantly disrupted in females. The edit(s) it produces are bisex dominant negatives. In both (b) and (c) the construct also functions to nullify the effect of the mutation at the RNA level by encoding an RNAi module or RNA editor and the target site on the chromosome with the construct has been recoded such that it is still functional but not recognised by the editor. (d) The construct is inserted into a haplo-sufficient gene needed in both males and females, but is inserted into a male-specific exon with the result that gene function is predominantly disrupted in males. The edit(s) it produces are female specific dominant negatives, achieved by targeting a female-specific exon of the same gene and the chromosome that the construct is on is protected from the edits because the target site has been recoded. (e) The construct is inserted into and disrupts a haplo-sufficient gene needed in females (left) or is inserted into and disrupts a haplo-sufficient gene needed in both sexes and has at its ends sequences that ensure it will be spliced out in males (right), with the result that gene function is predominantly disrupted in females. The genomic editor acts on the wild type allele located in a gene different from and tightly linked to the disrupted gene to produce a dominant negative mutation, a gain of function mutation, or a knock-out of a haplo-insufficient gene. (f) as in (e) where the target gene is female-specific. Note that in both (e) and (f) the target site on the chromosome with the construct has been recoded such that it is still functional but not recognised by the editor and the construct also functions to nullify the effect of the mutation at the RNA level (i.e. by encoding an RNAi module or RNA editor). (g) The construct is inserted into and disrupts a haplo-sufficient gene needed in females (left) or is inserted into and disrupts a haplo-sufficient gene needed in both sexes and has at its ends sequences that ensure it will be spliced out in males (right), with the result that gene function is predominantly disrupted in females. The genomic editor acts on the wild type allele located in a gene different from and tightly linked to the disrupted gene to produce a dominant negative, a gain of function mutation, or a knock-out of a haplo-insufficient gene, any of which causes lethality or sterility only in males. Note that in the single locus (d) and 2-locus linked (g) case, if the sex affected by the disruption is opposite to the sex affected by the edit, then it is not necessary for the construct to confer dominant protection against the edit. (h) The construct is inserted into and disrupts a haplo-sufficient gene needed in females (left) or is inserted into and disrupts a haplo-sufficient gene needed in both sexes and has at its ends sequences that ensure it will be spliced out in males (right), with the result that gene function is predominantly disrupted in females. The genomic editor acts on the wild type allele located in a gene distant from the disrupted gene to produce a dominant negative, a gain of function mutation, or a knock-out of a haplo-insufficient gene, any of which causes lethality or sterility only in males. In the case where the edit is made in a gene unlinked to the disruption, the construct must also encode a recoded version of the target gene such that it is resistant to editing whilst also restores function in individuals carrying a single copy of the dominant edit. (i) As (h) where the edits result in female-specific dominant sterility or lethality (rather than male-specific) and editing occurs only in males (e.g. Caso expression is controlled by a promoter only active in in males). Note that in d, g and h, if the words “female” and “male” are interchanged, then the strategy will typically still work, though with somewhat different dynamics.

FIG. 11 illustrates molecular configurations for various embodiments of the construct according to the invention, giving localised population suppression, non-localised partial suppression, or non-localised complete suppression, where the recessive effects of the construct are induced using a second gRNA within the construct. a) illustrates a molecular mechanism using a single HS^{ETDN} gene (analogous to the left-hand design in FIG. 2a). Here the construct encodes a genomic editor that i) targets the wildtype allele of the HS^{ETDN} gene to cause dominant lethality or sterility in both sexes and ii) targets an allele linked to the construct and located upstream from the dominant mutation to create a premature stop codon which causes recessive sterility or lethality in both sexes. The presence of the premature stop codon prevents the downstream dominant negative mutation from being expressed, and so individuals that inherit one copy of the construct, and the associated stop codon, and one wild type allele have nearly normal fitness (though they would be non-viable or sterile if homozygous for the construct and the associated stop codon). Specificity for the allele linked to the construct could be achieved by recoding the allele linked to the construct such that it is still functional but can be targeted the genome editor, whereas the WT allele would be immune. The genetic construct may also be configured such that the second gRNA induces recessive sterile or lethal mutations in a separate gene to that which is targeted by the first gRNA to induce the dominant sterile or lethal mutations. In which case, the construct can be either linked (analogous to that in FIG. 2b and illustrated in FIG. 11b) or unlinked (analogous to that in FIG. 2c and illustrated in FIG. 11c) to a HI gene or any gene in which it is possible to create dominant lethal or sterile mutations in both sexes or in only females targeted by the first gRNA, and in both cases the construct can be either linked or unlinked to the HS gene targeted by the second gRNA to induce the recessive lethal or sterile mutations.

### EXAMPLES

The inventors set out to identify a genetic construct that can be inserted into members of a species and released into a population, in order to suppress the population in an efficient manner over a period of time. As such, the inventors have developed a novel genetic construct, which offers increased efficiency for population suppression, owing to the ability of the construct to persist in the population over multiple generations. The inventors discovered that depending on the precise configuration, the genetic construct can have three different types of potentially useful impact when released into a target population: (i) localised suppression, (ii) non-localised partial suppression, or (iii) non-localised complete suppression.

### Materials and Methods

### Model

### 1. Overview

The following section describes the model used to simulate the suppression strategies. The model follows the deterministic structure developed by Burt and Deredec (2018, P. Roy. Soc. Biol. Sci. 285 (1883)), simulating a single, randomly-mating population of infinite size, with discrete, non-overlapping generations and two sexes. It is coded in Julia, a scientific programming language (Bezanson et al, 2012, arXiv: 12095142), and all simulations are performed within the Jupyter notebook interface (Kluyver et al, 2016, IOS Press). For the basic single-construct designs, the inventors modelled the release of a single genetic construct, which creates edits at a specific site in the genome and explored two scenarios. The first is that where the target site is located within the same locus as the construct and the second where the target site is located elsewhere in the genome. For the localised suppression strategies, the inventors also modelled a second construct, which allows for homing of the first and, potentially, itself. The inventors also simulated the release of multiple constructs, each with the same design as the simple single construct case, where each are located in and target separate loci from the first construct.

To model each of these scenarios, the inventors used a flexible simulator which can model multiple loci (n=L), each of which can have several alleles, one of which may contain a construct. The inventors modelled the population by tracking all possible individual genotypes containing all possible combinations of alleles across all loci being considered and allowed linkage between each pair of loci to vary. Depending on the design of the construct, it can influence gamete transmission by cleaving the wildtype (WT) allele of any of the loci being modelled, resulting in homing of the allele on the homologous chromosome or by mutating the wildtype allele. By varying genotype-specific fitness costs associated with host-gene disruption and editor-induced mutations, the inventors allowed constructs to be inserted into a range of different genes (e.g. haplo-sufficient, haplo-insufficient, etc.). The following sections describe how these processes are implemented in the model.

### 2. Gamete Transmission

Depending on the genotype, the probability of gamete transmission may be altered due to cleavage of the target site. This is modelled using a set of locus—(L) and sex—(G) specific parameters. Cleavage requires the presence of one of more constructs which collectively contain components capable of creating a site-specific cut (e.g. Cas9 and a gRNA) and the corresponding target allele, assuming the activity associated with each construct component is dosage independent. Cleavage of the WT at locus & in sex & occurs with probability

$${c}_{L}^{G} ,$$

and, in heterozygous for the target site, repair of the cleaved chromosome can occur by non-homologous end joining (NHEJ) with probability

$${j}_{L}^{G}$$

converting the WT to a cleavage-resistant mutant alleles, r1 and r2, with probability

$${P}_{L}^{G}$$

and

$$1 - {p}_{L}^{G} ,$$

respectively. Alternatively, repair can occur through homology-directed repair (HDR) in which homing occurs with probability

$$1 - {j}_{L}^{G} .$$

In the case where cleavage occurs at a locus homozygous for the WT allele, cleavage and subsequent mutation of each WT allele occurs with probability

$${m}_{L}^{G} ,$$

converting the WT to r1 or r2 alleles in a ratio

$${p}_{L}^{G} : 1 - {p}_{L}^{G} .$$

When more than one locus is being modelled, the inventors allow for recombination to occur between each pair of loci during gamete production. Assuming loci are ordered linearly along a chromosome (A to Z), r₁ describes the probability of recombination between locus i and i+1, requiring a total of r_{L-1} parameters, where L is the total number of loci being modelled. Recombination at different locations on the chromosome is assumed to occur independently (i.e. there is no interference) and loci on different chromosomes can be modelled with r=0.5. For reference, descriptions of all parameters included in the simulator can be found in Table 1.

### 3. Fitness Effects

The inventors allowed the fitness of a genotype relative to the WT to be affected by the disruption and/or editing of host gene. To model these fitness effects, for simplicity the inventors allowed for two distinct fitness profiles per locus, in which the first describes fitness due to insertion of a construct or by an r2 mutation (I), and the second describes fitness associated with an r1 mutant (R). The inventors applied this principle to all loci, modelling the fitness profiles at each using a set of locus- and sex-specific parameters. If W is the WT host gene, D is the allele containing a construct and r2 and r1 are cleavage-resistant mutant alleles, then the fitness's of each individual due to disruption of a host gene at locus L relative to the WT are:

$${W}_{L}^{G} / {W}_{L}^{G} = 1$$

$${W}_{L}^{G} / {D}_{L}^{G} or {w}_{L}^{G} / r {2}_{L}^{G} = 1 {h}_{I , L}^{G} {s}_{I , L}^{G}$$

$${D}_{L}^{G} / {D}_{L}^{G} or r {2}_{L}^{G} / r {2}_{L}^{G} = 1 - {s}_{I , L}^{G}$$

$${W}_{L}^{G} / r {1}_{L}^{G} = 1 - {h}_{R , L}^{G} {s}_{R , L}^{G}$$

$$r {1}_{L}^{G} / r {1}_{L}^{G} = 1 - {s}_{R , L}^{G}$$

$${D}_{L}^{G} / r {2}_{L}^{G} or r {2}_{L}^{G} / r {1}_{L}^{G} = 1 - {s}_{IR , L}^{G}$$

where

$${s}_{I , L}^{G} , {s}_{R , L}^{G} and {s}_{IR , L}^{G}$$

are selection coefficients, and

$${h}_{I , L}^{G} and {h}_{R , L}^{G}$$

are dominance coefficients, each of which differ depending on the locus and between sexes.

The fitness costs due to the edit can be mitigated if one or more constructs present in the genome carry a rescue component specific to the edited gene. To model this, the inventors counted the number of functional copies of locus L present in the genotype, including both WT and rescue copies. Where there are two functional alleles the relative fitness due to host-gene disruption is 1. Where there is a single functional allele the relative fitness due to host-gene disruption is

$$1 - {s}_{I , L}^{G} .$$

here the inventors made the assumption that the function of the WT and rescue are equivalent, and that there are no additional costs when there are more than two functional copies of a gene.

### 4. Population Biology

The model allows for simulation of a population through time assuming discrete, non-overlapping generations, and two life stages (juveniles and adults), where juvenile survival is density-dependent according to the Beverton-Holt model. In each generation, adult males and females produce gametes, during which recombination occurs between the two loci and, depending on the genotype, homing or mutation may occur in males or females. It is assumed that the population mates randomly, that females produce f (Nₘ/(Nₘ+bN_{eq})) fertilised eggs, allowing for the number of eggs laid to depend on the number of males present (Nₘ) when b is not equal to 0. Survival of the juveniles is density-dependent, such that the probability of surviving is

$${q}_{J} \frac{\text{?}}{a + z} ,$$

$$\text{?} \text{indicates text missing or illegible when filed}$$

where q_{J} is the density-independent probability the juvenile survives to adulthood, a determines the strength of density-dependent mortality and Z is the number of juveniles in the population. Here, all results are reported in terms of relative population sizes (compared to the pre-release equilibrium), therefore they are unaffected by the precise value of α. The intrinsic rate of increase (Rₘ) of the wild-type population when

$$b = 0 is \frac{\text{?}}{2} .$$

$$\text{?} \text{indicates text missing or illegible when filed}$$

For simplicity, all genotype-dependent fitness costs associated with host gene disruption or construct activity are assumed to affect survival after density-dependent juvenile mortality and before censusing (e.g., as if pupae die).

TABLE 1
Model parameters

| | |
|---|---|
| Parameter | Description |

Gamete production

| | |
|---|---|
| c_{L}^{G} | Probability of cleavage in individuals heterozygous |
| | for the target site |
| j_{L}^{G} | Probability of end-joining given cleavage above. |
| | Set equal to 1 in all simulations. |
| m_{L}^{G} | Probability of mutation in individuals homozygous |
| | for the target site |
| p_{L}^{G} | Probability of an r1 mutant being produced |
| | through end-joining. |
| | Set equal to 1 in all simulations. |
| rᵢ | Recombination between locus i and i + 1 |

Locus-disruption fitness costs

| | |
|---|---|
| s_{I, L}^{G} | Fitness cost for target site disruption by insertion |
| | of a construct or r1 mutant |
| s_{R, L}^{G} | Fitness cost for target site disruption by r2 mutant |
| h_{I, L}^{G} | Dominance coefficient for target site disruption by |
| | insertion of a construct or r1 mutants |
| h_{R, L}^{G} | Dominance coefficient for target site |
| | disruption by r2 mutants |
| s_{IR, L}^{G} | Fitness cost for heterozygous for the two |
| | types of disruption (construct or r2 and r1) |

Population dynamics

| | |
|---|---|
| Rₘ | Intrinsic rate of population increase. |
| | Set equal to 6 in all simulations. |
| b | Male limitation parameter. Set equal to 0.001 |
| | in all simulations. |

L refers to the locus and G to the sex of the individual.

### Screens

The inventors first simulated the release of a range of different constructs based on the single locus design, where the construct can cause a phenotypic effect by disrupting the function of its insertion site and also by generating mutations at a position within the same gene. Here, the inventors modelled a single autosomal locus with three alleles, the wild type allele, the transgenic construct inserted into and disrupting the wild type allele and the non-functional mutation generated by the construct. The inventors modelled scenarios where the construct can be inserted into a gene causing dominant

$$( {s}_{I}^{G} = 1 , {h}_{I}^{G} = 1 )$$

or recessive

$$( {s}_{I}^{G} = 1 , {h}_{I}^{G} = 0 )$$

lethality in adults or be neutral

$$( {s}_{I}^{G} = 0 , {h}_{I}^{G} = 0 ) ,$$

and the gene can be sex-specific, where G=F in females and G=M in males. Similarly, the inventors allowed the phenotypic effects of the mutation to be dominant

$$( {s}_{R}^{G} = 1 , {h}_{R}^{G} = 1 ) ,$$

recessive

$$( {s}_{R}^{G} = 1 , {h}_{R}^{G} = 0 )$$

or neutral

$$( {s}_{R}^{G} = 0 , {h}_{R}^{G} = 0 )$$

either sex or both. The inventors also allowed for the fitness effects in individuals heterozygous for the construct and mutation alleles to be neutral

$$( {s}_{IR}^{G} = 0 )$$

or cause lethality/sterility

$$( {s}_{IR}^{G} = 1 ) ,$$

again allowing these to differ between sexes. Finally, the inventors allowed for sex-specific mutation based on the expression activity of the construct, where mutation can occur only in males (c^{m}=1, c^{F}=0), females (c^{m}=0, c^{F}=1) or in both sexes (c^{m}=1, c^{F}=1). All end joining rates (j^{G}) were equal to 1, ensuring all cleaved alleles resulted in a mutation and that homing of the construct was not possible. They also set p^{G}=1, ensuring all mutations generated by the construct were of the same type in terms of their fitness effects, and that their fitness could differ from that of the construct itself.

The inventors performed similar simulations assuming that the edits made by the constructs were located elsewhere in the genome and that the construct contained a recoded version of the target locus. Here, the inventors modelled two loci, each with two alleles and assumed the loci were unlinked (r=0.5). At the first locus (A) the two alleles included the wild type and the transgenic construct, and at the second (B) the inventors included the wild type and the mutant generated by the construct. Again, the inventors allowed the fitness effects of the construct to vary, causing dominant

$$( {s}_{I , A}^{G} = 1 , {h}_{I , A}^{G} = 1 )$$

or recessive

$$( {s}_{I , A}^{G} = 1 , {h}_{I , A}^{G} = 0 )$$

lethality in adults or to be neutral

$$( {s}_{I , A}^{G} = 0 , {h}_{I , A}^{G} = 0 ) ,$$

and be sex-specific, where G=F in females and G=M in males. Similarly, the inventors allowed the phenotypic effects of the mutation at locus B to be dominant

$$( {s}_{I , B}^{G} = 1 , {h}_{I , B}^{G} = 1 )$$

or neutral

$$( {s}_{I , B}^{G} = 0 , {h}_{I , B}^{G} = 0 )$$

in either sex or both. Finally, the inventors allowed for sex-specific cleavage at the B locus based on the expression activity of the construct, where cleavage can occur only in males

$$( {c}_{B}^{m} = 1 , {c}_{B}^{f} = 0 ) ,$$

females

$$( {c}_{B}^{m} = 0 , {c}_{B}^{f} = 1 )$$

or in both sexes

$$( {c}_{B}^{m} = 1 , {c}_{B}^{f} = 1 ) .$$

The inventors assumed the probability of cleavage of a WT allele in heterozygotes and homozygotes for the WT allele was equal

$$( {m}_{L}^{G} = {c}_{L}^{G} ) .$$

The inventors also assumed that the recoded version of the target locus was functionally equivalent to the WT allele, i.e. in the presence of two edited alleles, one copy of a construct was sufficient to rescue edits with a recessive phenotype but insufficient to rescue edits with a dominant phenotype.

For all possible combinations of these parameters sets for the two different designs, the inventors simulated single releases of male heterozygotes released at 50% of the initial male population, monitoring the relative number of females for 200 generations. The inventors categorised the results into three groups based on their dynamics. Strategies giving less than 50% suppression were investigated for how they behaved with repeated releases, simulating releases of 5% each generation. Those where the relative female population size was <1% were investigated further for complete elimination and those where it was between 1% and 50% were investigated further for partial suppression. Within this final group, the inventors repeated the simulations using less than ideal parameters to identify those most robust to fluctuations in fitness and cleavage parameter values (5% fitness costs in heterozygotes when either the construct or the target site mutant were recessive and 80% cleavage efficiency).

### Identifying Target Genes

A reasonable course of action to implement the invention for localised suppression would be to first sequence the genome of the target species (if it does not exist already), and then to look for homologs of known genes with the required properties.

For example, homologs of the doublesex gene would be suitable.

In addition, any gene that is haplo-sufficient yet editable to give a dominant sterile or lethal phenotype can also be used. A search of FlyBase indicates there are 45 genes with both dominant lethal and recessive lethal mutations, including 5-HT2A, Antp, BicD, cact, chic, Col4a1, crn, cype, dare, dl, dpp, Fs(2)Ket, gro, hb, hh, hop, Hsc70-3, Hsc70-4, ken, l(1)10Ad, l(1)10Ae, l(2)25Ca, l(2)40Ff, l(2)46Fb, l(2)DTS18SP, l(2)DTS19, l(2)DTS20, l(2)DTS6, l(2)DTS8, l(2)DTS9, l(2)M167, lt, M(3)80, nos, Prosbeta6, puc, rl, Scr, snf, stmA, Sxl, tkv, Tl, tor, wupA.

In order to confirm the recessive phenotype, one could then experimentally knock out the target gene in the target organism, to confirm the recessive phenotype (bisex lethality or sterility), and create truncation mutations (by introducing premature stop codons) in the last two exons, or the last third of gene, or within a certain number of bp from the end, or other edits suggested by the homology and confirm the dominant phenotype (female or bisex lethality or sterility).

If the two phenotypes have been confirmed, then one would create the inventive haplotype with a disrupted or deleted haplo-sufficient gene and the editor, ensuring the haplotype either does not get edited (e.g., because the recognition site(s) for the editor do not exist) or the edit is not expressed (e.g., because it occurs after a stop codon).

Alternatively, if the first experiments confirm the dominant phenotype of the edit but not the recessive phenotype of the knock-out, then one could examine nearby genes to see if there are any that are homologous to haplo-sufficient essential genes in Drosophila (or other model organisms), and then do a knockout to confirm the recessive lethal or sterile phenotype. If this is confirmed, then one would create a haplotype with the disrupted haplo-sufficient gene, the editor, and a target gene that has been recoded (or deleted) such that it is not recognised by the editor. In this case it may be necessary to choose an editor that does not stimulate recombinational repair, e.g. a base editor or reverse prime editor, or one that cleaves the target site to produce 3′ overhangs. Additionally, it may be appropriate to reduce the likelihood of recombination breaking up the haplotype, such as by inverting some or all of the sequence.

Alternatively, if the first experiments confirm the dominant phenotype of the edit but there is no nearby gene that is haplo-sufficient essential, then one could search more distantly in the genome sequence for homologs of known essential haplo-sufficient genes (e.g., there are 818 genes in FlyBase annotated as having an amorphic or loss of function allele giving a recessive lethal phenotype). One would choose one such gene, confirm the knock-out has a recessive sterile or lethal phenotype, and then make the inventive haplotype consisting of the disrupted gene, the editor, and a rescue gene that suppresses the dominant edit (e.g., by encoding one or more copies of the target gene, recoded to not be recognised by the editor, or by encoding functions that nullify the edit at the RNA level, such as by RNAi or RNA editing).

Alternatively, if dominant mutations cannot be found in homologs of the haplo-sufficient genes listed above, then one could look for homologs of known haplo-insufficient genes. Cook et al. (2012: Table 2) list 43 genes in Drosophila melanogaster that are haplolethal or haplosterile, plus another six regions of the genome that appear to have a haplolethal or haplosterile within them but the exact gene had not yet been identified. The majority of these encode protein components of cytoplasmic ribosomes (Rp genes) or translation initiation factors (eIF genes) and are associated with the Minute syndrome, a characteristic set of phenotypes including short, thin bristles and slower development. Thus, one preferred embodiment of the invention has the editor targeting a homolog of one of these genes, or of another cytoplasmic ribosomal protein or translation initiation factor.

Other classes of genes showing haplo-insufficiency (and that might be lethal in nature if not in the lab) include those that encode the muscle components actin (Act88F), myosin (Mhc and Mlc2) and tropomyosin (Tm2), as well as a group of closely linked, muscle-related genes regulated by a haplolethal sequence within an intron of the Troponin I (wupA) gene. This subset may also include Hdl, which may correspond to Troponin T (up). Like the ribosomal protein genes, these genes may be particularly dosage sensitive because muscle assembly requires minimal levels or a particular stoichiometry of component proteins. Other definable subsets encode homeodomain proteins (Abd-B, Dll, Scr and Ubx), Notch pathway components (Dl, H and N), Polycomb group repressor proteins (Pc and Pcl), apoptosis regulators (lok and p53) and melanin biosynthetic enzymes (b and e).

One would then confirm experimentally that knock-out of the homolog gives a dominant sterility or lethality phenotype, either in one sex or both sexes (e.g., by causing a premature stop codon). One would then proceed as above, looking first for nearby haplo-sufficient genes, or, alternatively, for more distant haplo-sufficient genes, and construct the inventive haplotype accordingly.

If the goal is a self-spreading intervention that gives either partial or complete control, then the combination of the fitness effects of the gene disruption will be different, but the analogous procedures of using homology and experiments can be used to build the inventive haplotype. For example, if the goal is a self-spreading intervention giving complete suppression, then one of the combinations to achieve this is for the disrupted haplo-sufficient gene to have a sterile or lethal phenotype only in females, and for the edit to have a dominant effect only in females (FIG. 5). Again, homologs of the doublesex gene may be suitable targets, if the recessive disruption and dominant edit are confined to the female-specific exon (FIG. 6a right and 6b left). Alternatively, homologs of Drosophila genes that are female-specific haplo-sufficient and editable to being a dominant female-specific negative, including homologs of the Drosophila genes ovo, dorsal, torso, easter, or Toll, may also be suitable (FIG. 6a left).

If the goal is to have a self-spreading intervention giving partial suppression, then the construct may also encode functions that nullify the edit in trans at the RNA level, for example by RNAi or RNA editing.

For some embodiments the gene that is disrupted, while essential in nature, is not essential in the lab, in order to facilitate rearing a homozygous pure-breeding line. As such, the gene disruption may be auxotrophic and able to be compensated for by a dietary supplement. The gene may be involved in the biosynthesis of purine, pyrimidines, fatty acids, or rescuable by dietary supplement with these factors, or fructose or lineolate. The gene may be a homolog of the Drosophila genes rudimentary, rudimentary-like, Dhod, ade2, ade3, ade4, ade5, bur, Pgd, or SREBP.

If the target species is a mammal, then similar approaches may be taken by those skilled in the art using publicly available data (e.g., in the MGI or OMIM databases) to identify suitable genes as queries to look for homologs in the target species. Already lists exist of mouse genes likely to be haplo-insufficient or intolerant of loss of function (e.g., https://search.clinicalgenome.org/kb/curations) and of human genes that can mutate to dominant negative or dominant gain of function alleles (Coban-Akdemir et al. 2018 Am J Hum Genet 103:171-187). This reference emphasises how many mutations that produce dominant negative or dominant gain of function alleles are premature stop codons in positions where the transcript will not be subject to nonsense mediated decay.

### Results

### Example 1—Localised Suppression

### 1.1 1- and 2-Locus Screens

The single-locus screen for strategies giving efficient localised suppression in 50 generations with repeated 5% release rates revealed three strategies, two of which also appeared in the 2-locus screen. As shown in FIG. 1, the 2-locus screen was more constrained in terms of allowable parameter combinations than the 1-locus screen and the third strategy was not included in the allowable strategy set for the 2-locus screens. FIG. 1 also shows the expected decline in population size with these strategies, under certain idealised conditions, compared to release of sterile males, which is a close comparator method that has been widely used to control some pests. As can be seen, the constructs according to the invention can be substantially more efficient.

The strategies involve releases of a construct, which either causes or is associated with a recessive lethal or sterile phenotype in both males and females. The construct creates either bisex or female-specific dominant mutations, or (in the 1-locus case) a female-specific dominant phenotype with male-specific recessive phenotype.

### 1.2 Molecular Configurations

Three alternative proposed molecular configurations, applicable to all three strategies, are shown in FIG. 2, where an edit can made in (a) the same gene, (b) a different gene tightly linked to the construct, or (c) an unlinked gene, each of which give the same dynamics as shown in FIG. 1 when simulated with idealised parameters. The construct comprises a nucleic acid sequence which encodes a genomic editor, which could be any of: a transcription activator-like effector nuclease (TALEN) genomic editor, Zinc finger nuclease (ZFN) genomic editor, or a CRISPR-based genomic editor, such as CRISPR-Cas9 used with one or more guide RNA (gRNA) sequence. For illustration purposes, the figures show Cas9-gRNA though the skilled person will realise that the invention could be applied using any other genome editing technique.

The sequence encoding the genomic editor is flanked by nucleic acid sequences which allow for homologous recombination and therefore integration into a haplo-sufficient gene (HS). As such, the construct may be inserted into the genome in such a way that it disrupts a haplo-sufficient gene that is needed for survival or reproduction of males and females, as shown in FIG. 2a-c.

Alternatively, it may be associated with a mutation that causes recessive lethality or sterility, but little effect on fitness when heterozygous with a wild type allele. For example, the invention may consist of a natural gene in the target organism being replaced by one in which a recessive lethal or sterile mutation has been introduced and that also contains the genome editor in an intron, either natural or synthetic. Alternatively, the editor may be tightly linked to the gene into which the recessive mutation has been introduced.

In summary, the three required features of the design for localised suppression are:
a) the genetic construct causes recessive sterility or lethality in both sexes.
b) the genetic construct creates mutations which cause dominant sterility or lethality in both sexes or only females.
c) the genetic construct provides protection against a single copy of the dominant mutations it creates.

FIG. 2 shows example molecular configurations that can be used for localised population suppression.

FIG. 2a left shows two chromosomes of a diploid individual, one chromosome (top) with a haplo-sufficient gene needed in both sexes that has been disrupted by the insertion of a genome editor (denoted by the Cas9 and gRNA boxes), which introduces a premature stop codon in the HS gene, and one chromosome (bottom) with a wild type allele of the haplo-sufficient gene. The arrows indicate that in organisms that are heterozygous for these two alleles, the editor acts on a target sequence downstream of the insertion site of the editor on both the chromosomes. This editor produces dominant mutations that, in a wild type genetic background, cause lethality or sterility. The mutation on the chromosome with the construct is not expressed because of the premature stop codon. The HS gene must therefore be one in which it is possible to introduce a dominant negative (DN) or a dominant gain of function mutation, and therefore it is labelled as HS^{ETDN} (standing for haplo-sufficient, editable to dominant negative; here and elsewhere dominant negative and dominant gain of function mutations are combined under the label ‘dominant negative’). Numerous genes have been reported in which loss of function mutations are recessive lethal or sterile and dominant lethal or sterile mutations have been observed, and, more generally, there are many genes in which both recessive and dominant lethal or sterile mutations have been reported (see earlier in description). Control sequences that determine the tissue specificity of editor expression are chosen such that the editor is expressed in the germ line, such that zygotes that are heterozygous for a construct-bearing allele and a wild type allele should have normal or near normal fitness, but transmit the edited allele to a large fraction (approaching 50%) of their progeny. The configuration shown is illustrative, and can be changed in several ways while keeping to the invention. (1) Many variants of the editor may be considered. The editor may have more than one gRNA (to increase efficacy and/or reduce the likelihood of resistance evolving). The editor may not use Cas9, but instead use a Cas9-derived protein, to allow DNA nicking, base editing, prime editing, or some other type of edit. Alternatively, it may use Cpf1. Alternatively, it may not be CRISPR-based, and instead use an architecture based on

TALENs or ZFNs. (2) The insertion site for the construct may be downstream rather than upstream of the target sequence recognised by the editor; in this case the target site on the chromosome with the construct should be modified so it is not recognised by the editor. (3) The construct is shown as being inserted into the HS gene, but some or all of the HS gene may be deleted (becoming more like a gene replacement), or the construct may be inserted outside of but closely linked to a disrupted allele of the HS gene. (4) Optionally, the construct may also encode a marker, such as a fluorescent protein, to aid tracking of the construct (not shown).

FIG. 2a right shows a molecular configuration in which the genetic construct (shown here as Cas9 and gRNA as an example) is inserted into and disrupts a haplo-sufficient gene needed for viability or fertility in both sexes (HS), and the genomic editor targets a sequence in a female-specific exon of that gene (pink) to make dominant female-specific lethal or sterile mutations (arrows). Thin pink and blue lines show the sex-specific splicing pattern of the gene; for simplicity they are only shown on the top chromosome, but also apply to the bottom (wild type) allele. Homologs of the Drosophila gene doublesex may be appropriate genes to use for this configuration, as loss of function mutations are recessive sterile in both sexes and they have a female-specific exon in which dominant female sterile mutations can be created. The same variations as described for FIG. 2a left also apply here (nature of the editor, relative position of construct and target site, the option of deleting much of the HS gene as part of construct insertion, etc).

FIG. 2b shows a molecular configuration in which the genetic construct (shown here as Cas9 and gRNA as an example) is inserted into and disrupts a haplo-sufficient gene needed for viability or fertility in both sexes (HS), and the genomic editor targets a sequence in a gene located in a separate gene to the integration site of the genetic construct, but which is tightly linked to it (left; HI and right; Gene^{ETDN}). This editor produces dominant mutations that, in a wild type genetic background, cause lethality or sterility. Note that the arrows show that the editor acts only to edit the target gene located on the chromosome which does not contain the genetic construct. The genomic editor could be configured to create a knock-out mutation in a wild type allele of a haplo-insufficient gene (left; HI). In this case, the target site located on the chromosome containing the genetic construct comprises a recoded version of the target site, such that it is functional but not recognised by the editor (rHI). Alternatively, the genomic editor could be configured to create dominant negative mutations in any gene (right; Gene^{ETDN}), in which case the chromosome containing the genetic construct can confer resistance to editing by containing a deletion of the target site, or a deletion of the entire target gene (indicated by a hashed rGene^{ETDN}). Variations (1), (3) and (4) of FIG. 2a left also apply here (nature of the editor, the option of deleting much of the HS gene as part of construct insertion and inclusion of a marker). For illustration, FIG. 2b left and right show the editor acting upon a target sequence located in the separate gene downstream of the insertion site of the editor, however, the target site could alternatively be located in a gene upstream of the integration site of the editor.

FIG. 2c shows a molecular configuration in which the genetic construct (shown here as Cas9 and gRNA as an example) is inserted into and disrupts a haplo-sufficient gene needed for viability or fertility in both sexes (HS), and the editor targets a sequence located in a separate gene that is distant from the integration site of the genetic construct (left; HI and right; Gene^{ETDN}). The arrows indicate that in organisms that carry a construct, the editor acts on a target sequence on both the chromosomes to produce dominant mutations that, in a wild type genetic background, cause lethality or sterility. The genomic editor could be configured to create a knock-out mutation in a wild type allele of a haplo-insufficient gene (left; HI) or to create dominant negative mutations in any gene (right; Gene^{ETDN}). In both cases, the construct may also encode a module which provides the organism protection against one copy of an edited allele, but not two copies. If the edit is a knock-out of a haplo-insufficient gene (left), the rescue module could encode a recoded copy of the target gene which the editor is unable to recognise and edit, but which retains the function of the wild type target allele. Alternatively, if the edit is a dominant negative mutation, the rescue copy of the construct could be configured to have higher expression levels to dilute out the effect of the mutation. Variations (1), (3) and (4) of FIG. 2a left also apply here (nature of the editor, the option of deleting much of the HS gene as part of construct insertion and inclusion of a marker).

For illustration, the genetic constructs in FIGS. 2a-c have been depicted as being inserted into and causing a disruption of the haplo-sufficient gene. FIG. 2d left and middle illustrate how the construct may alternatively be configured to integrate into a disrupted allele of the haplo-sufficient gene, where the disruption may be due to the introduction of a premature stop codon (grey) or a deletion of all or part of the gene (hashed box) (d; left and middle). Alternatively, the genetic construct may integrate into a location outside of, but near to, a disrupted allele (d; right). Although throughout this document all constructs are depicted to cause the disruption (as shown, for example, in FIG. 2a left), all designs can also be built where the editor is non-causally associated with a disruption (as shown in FIG. 2d).

The recessive lethal or sterile mutation may be chosen such that it can be masked in the lab and in the released individuals, and the editor or dominant edit designed such that it is repressible in the lab, so that a pure-breeding strain can be produced, reducing the costs of production and allowing homozygous individuals to be released, increasing the impact of those releases (or allowing them to be smaller).

### 1.3 Increasing Efficiency

The organisms carrying the construct may also carry a second construct that causes the frequency of the primary construct to increase. For example, the second construct might encode a gRNA that allows the first construct to home, giving it a temporary boost in frequency, making it even more efficient while still staying localised (FIG. 3).

Referring to FIG. 3a, there is shown an example molecular configuration of a two-construct design in which the genetic construct illustrated in FIG. 2a left (shown here as the right-hand side construct) is paired with a second construct (left-hand side construct) containing a gRNA—inserted into a neutral locus (Ntrl)—targeting the wild-type sequence in which the first construct is inserted. The orange arrows show that the editor acts upon target sequences located downstream of the insertion site of the editor on both the chromosomes to generate dominant mutations which cause sterility or lethality in a wild type background, as illustrated in FIG. 2a. Alternatively, the target site on the chromosome with the construct may be independently modified to produce a dominant negative mutation (which is not expressed due to the premature stop codon introduced by the construct), which then homes across to the chromosome without the construct when that chromosome is cleaved by the editor. The green arrow indicates that in organisms that are heterozygous for the right-hand side construct and which carry at least one copy of the left-hand side construct, the sequence at the insertion site of the construct on the wild type allele is cleaved by the Caso complex produced from the gRNA encoded by the left-hand construct and the Cas9 encoded by the right-hand construct, causing the right-hand construct to home (be copied over to the opposite chromosome) using the cells natural homology-directed repair mechanism. If the construct is not causally responsible for the gene disruption (as in the configurations of FIG. 2d), then the modification that is responsible should co-home with the construct.

In an alternative configuration, the left-hand side construct could encode its own Cas9 or other RNA-guided nuclease. Note that a second construct containing a booster gRNA can be used with any of the molecular configurations shown in FIG. 2 and will have an effect similar to increasing the numbers released.

Alternatively, the second construct may encode two gRNAs, one allowing the primary construct to home and the other allowing the second construct to home in the presence of the primary one, creating a double drive which may be expected to spread to additional populations, and whose spread may be controlled by exploiting pre-existing sequence differences between target and non-target populations (FIG. 4).

Referring to FIG. 4a, there is shown an example molecular configuration of a double drive in which the genetic construct illustrated in FIG. 2a left (shown here as the a construct) is paired with a second construct (B) encoding two gRNAs, one targeting the insertion site of the α construct (green) and one targeting the wild-type allele of its own insertion site (grey). The orange arrows show that the editor acts upon target sequences located downstream of the insertion site of the editor on both the chromosomes to generate dominant mutations which cause sterility or lethality in a wild type background, as illustrated in FIG. 2a. Alternatively, the target site on the chromosome with the construct may be independently modified to produce a dominant negative mutation (which is not expressed due to the premature stop codon introduced by the construct), which then homes across to the chromosome without the construct when that chromosome is cleaved by the editor. In addition, the green and grey arrow indicate that in organisms that are heterozygous for the a construct and also heterozygous for the β construct, the sequence at the insertion site of each construct on the wild type alleles is cleaved by the Caso complex produced from the gRNAs encoded by B and the Cas9 encoded by the α construct, resulting in homing of both constructs. Homing of each construct individually can also occur in individuals homozygote for the other construct. If the β construct is inserted into a differentiated site (diff), where some chromosomes have a sequence recognised by the grey gRNA and some do not (and therefore are resistant to cleavage), and the frequency of the two types of chromosomes varies among populations, localisation of the construct and the population suppression can be achieved as long as the frequency of resistance is sufficiently low in the target population, allowing both constructs to spread, and high in the non-target population, preventing the β construct from spreading. In an alternative configuration, the β construct could encode its own Cas9 or other RNA-guided nuclease, allowing homing of β in the absence of α. The β construct could be paired with any of the molecular configurations shown in FIG. 2 to create a double drive with a similar effect.

Alternative methods of boosting the primary construct may require that it contain additional elements; for example, the primary construct may carry a rescue construct that masks the effect of mutations produced by the second construct. Further construct(s) may also be included which cause the second construct to increase in frequency.

The inventors also propose an alternative molecular design for achieving feature a) of FIG. 2, where the construct is configured to integrate into a location outside of a haplo-sufficient gene (either near to or more distantly linked to) and designed to induce mutations in the haplo-sufficient gene which cause recessive sterility or lethality, by, for example, including within the construct a second gRNA targeting the haplo-sufficient gene (see FIG. 11).

As illustrated in FIG. 11, localised population suppression (or non-localised partial or complete suppression) can be achieved with the genetic construct according to the invention, where the recessive effects of the construct are induced using a second gRNA within the construct. FIG. 11a illustrates a molecular mechanism using a single HS^{ETDN} gene (analogous to the left-hand design in FIG. 2a). Here the construct (shown as Cas9 and two gRNAs) encodes a genomic editor that i) targets the wildtype allele of the HS^{ETDN} gene to cause dominant lethality or sterility in both sexes and ii) targets an allele linked to the construct and located upstream from the dominant mutation to create a premature stop codon which causes recessive sterility or lethality in both sexes. The presence of the premature stop codon prevents the downstream dominant negative mutation from being expressed, and so individuals that inherit one copy of the construct, and the associated stop codon, and one wild type allele have nearly normal fitness (though they would be non-viable or sterile if homozygous for the construct and the associated stop codon). Specificity for the allele linked to the construct could be achieved by recoding the allele linked to the construct such that it is still functional but can be targeted the genome editor, whereas the WT allele would be immune.

As shown in FIGS. 11b and 11c, the genetic construct according to the invention may also be configured to induce recessive sterile or lethal mutations in a second gene, separate to that which is targeted to induce the dominant sterile or lethal mutations. In which case, the construct can be either linked (analogous to that in FIG. 2b and illustrated in FIG. 11b) or unlinked (analogous to that in FIG. 2c and illustrated in FIG. 11c) to a HI gene or any gene in which it is possible to create dominant lethal or sterile mutations in both sexes or in only females, and in both cases the construct can either linked or unlinked to the HS gene targeted to induce the recessive lethal or sterile mutations.

### Example 2—Full Suppression

### 2.1 1-Locus Screen

The 1-locus screen revealed 12 strategies that would give complete elimination after a single release (FIG. 5). For this case again, the construct is a recessive sterile or lethal and the edit is dominant, and the allele with the construct is protected from the dominant edit because of a premature stop codon, or the target site has been recoded or removed, but the sex-specificity is different than with localised control. FIG. 5 also shows time series simulations of relative female population size after a single release of constructs designed to use a single locus when released in heterozygotes at 50% of the initial male population size.

If the construct and gene disruption affect only one sex, then either the edit should affect the same sex as the construct (fFF, mMM), or the edit should be bisex (fBB, mBB), or the edit should be bisex with partial protection in trans, such that the same sex is affected in the construct/edit heterozygote as in the construct/construct homozygote (fBF,mBM). In this last case, full suppression is achievable even when editing occurs in only one sex, as long as that sex is the one which experiences the recessive fitness effects due to the construct insertion. Alternatively, if the construct and gene disruption affect both sexes, then the edit should also be bisex, and there should be sex-specific protection in trans (i.e., the construct/edit heterozygote is sterile/lethal in only one sex) (bBF, bBM).

When there is some cost for individual heterozygotes for either the construct or edit when otherwise they would be completely recessive

$$( {h}_{L}^{G} = 0.05 )$$

and editing is suboptimal

$$( {c}_{L}^{G} = 0.8 ) ,$$

only three designs are able achieve more than 99% suppression. These include strategies 3, 5, and 9 (fBFB, fBBB and fFmFB). If editing is only possible to achieve in one sex, then strategies 7 and 8 are suitable.

### 2.2 Molecular Configurations

These requirements can be met by the appropriate combination of using genes required only in one sex, exons that are required only in one sex (due to sex-specific splicing), by incorporating sequences into the construct that ensure it is spliced out in one sex but not the other, and by modifying control regions on a gene so it is expressed only in one sex or to increase expression to give partial (sex-specific) protection (FIG. 6).

Referring to FIGS. 6a and b, there is shown example single locus molecular configurations that can be used for full self-sustaining suppression. In each, if the words “female” and “male” are interchanged, then the strategy will still work, although with different dynamics. FIG. 6a left shows an example design for strategy 1, fFF, (and 2 [mMM] if sexes are reversed) of FIG. 5. Here the construct (shown here as Cas9 and gRNA), is inserted into and disrupts a female-specific haplo-sufficient gene (HS^{ETDN}, pink). The arrows indicate that in organisms that are heterozygous for the genomic construct and a wild type allele of the female-specific HS gene, the editor acts on a target sequence downstream of the insertion site of the editor on both chromosomes to produce dominant negative mutations that, in a wild type genetic background, cause lethality or sterility. Alternatively, as illustrated in 6a right, the construct can be inserted into a female-specific exon (HS^{ETDN}, pink) of a bisex haplo-sufficient gene (HS, grey), such that gene function is predominantly disrupted in females, and the genomic editor configured to create dominant negative mutations at a target site downstream of the disruption within the same sex-specific exon, or another sex-specific intron of the same gene. Thin pink and blue lines show the sex-specific splicing pattern of the gene; for simplicity, they are only shown on the top chromosome, but also apply to the bottom (wild type) allele. In both cases (left and right), where the edit is downstream of the disruption, the mutation on the chromosome with the construct is not expressed because of the premature stop codon, providing protection for the construct. However, the target site could also be located upstream of the disruption, in which case the target site on the chromosome containing the construct must be modified or removed so that it is not recognised by the editor.

FIG. 6b illustrates example molecular designs for strategy 3, fBB, (and 4 [mBB] if sexes are reversed), where the disruption is sex-specific and the dominant negative mutations created by the editor affect both sexes. Left: The construct is inserted into and disrupts a female-specific exon (HS, pink) of a haplo-sufficient gene and targets a second exon of the same gene expressed in both sexes (HS^{ETDN}, grey). Right: Alternatively, the construct is inserted into a haplo-sufficient gene required for both sexes (HS^{ETDN}, grey), but has splicing control sequences (not shown) which result in it being spliced out in males, such that the disruption predominantly effects females. In both cases, the target site on the chromosome with the construct has been recoded and so is not recognised by the editor (rHS^{ETDN}; no orange arrow).

Strategies 3 and 4 can also be implemented using two separate loci (FIG. 6c), in which the genomic editor is configured to generate dominant mutations in a gene tightly linked to the gene disrupted by the construct (indicated by the arrows). The construct can be inserted into and disrupt a female specific haplo-sufficient gene (left; HS, pink) or a haplo-sufficient gene required in both sexes (HS, grey) but contain sequences which ensure it is spliced out in males (right). The genomic editor can be designed to create a knock-out mutation of a haplo-insufficient (HI) gene or to produce dominant negative mutations of another gene (Gene^{ETDN}, grey). In both cases, the target site on the chromosome containing the construct may be recoded such that it is protected from the editor (rHI/rGene^{ETDN}; no orange arrows).

FIG. 6d illustrates example configurations of strategy 11 of FIG. 5 (and 12 if sexes are reversed). Here the construct is inserted into and disrupts a haplo-sufficient gene needed in both males and females (HS, grey) and the editor creates dominant mutations in a gene needed in both sexes and closely linked to the disrupted gene. Left: the editor targets a gene to create dominant negative mutations. The chromosome containing the construct is modified such that the gene containing the target site is not recognised by the editor (rGene^{ETDN}), yet is functional, and has increased expression in males, such that only female heterozygotes for the edit and the construct are lethal or sterile (blue arrow indicating male-specific enhancement). Alternatively, rather than increasing expression of the modified gene in males, the construct could also encode a module which is expressed only in males and acts to nullify the effects of the dominant negative mutation. This could be an RNAeditor (RNAe), which reverts the RNA transcribed from the edited gene back to the wild-type, or an RNA interference (RNAi) module which degrades it. Right: Alternatively, the editor is configured to create knock-out mutations in a closely linked haplo-insufficient gene required in both sexes (HI, grey). The chromosome containing the construct is modified such that the gene containing the target site is not recognised by the editor (rHI), yet is functional, and contains a second copy of the recoded target gene which also contains sequences which ensure it is only expressed in males, ensuring males heterozygous for the construct and edit have normal fitness and females are sterile or lethal. Male-specific expression could be achieved by use of an appropriate promoter, as shown here (blue arrow), or by inserting an intron that will be spliced out only in males, and introduce a premature stop codon in females. Alternatively, rather than incorporating a second copy of the modified gene expressed only in males, the construct could contain a module which nullifies the effects of the haplo-insufficient knockout, such as an RNA editor.

### 2.3 2-Locus Screen

These requirements can also be met using two closely linked loci or two distantly linked or unlinked loci. Designs 1~4 can also be implemented using a two-locus model where the edit is made at an unlinked locus and the construct fully rescues the dominant fitness effects of the edit (dynamics are identical as shown in FIG. 7). If editing is only possible to achieve in one sex, then strategies 7 and 8 are still useful, and also more efficient than when implemented using a single locus case, as are 13 and 14.

### 2.4 Increasing Efficiency

The rate at which the construct spreads through the population and suppresses the population could be increased if the construct also catalysed its own homing reaction. Even low rates of homing could increase the rate (or reduce the release rate required to get suppression in a specific timeframe).

### Example 3—Partial Suppression

Another use of the invention can be to provide for non-localised (self-spreading) partial control. This may be useful if the end goal of the control programme is partial control, or if the end goal is complete control, but there is a desire to approach that in a step-wise manner as part of a risk mitigation strategy. The inventors are not aware of any other strategy having been proposed that gives self-spreading partial control when fitness effects and editing rates are idealised (i.e., 0 or 1).

### 3.1 1-Locus Screen

The single locus screen identified 26 strategies where the equilibrium level of suppression falls between 50% and 99% (FIG. 8). For this case, the construct can be associated with recessive sterile or lethal effects in both sexes or only one. If the effects are bisex (strategies 1-4 of FIG. 8), the construct must provide dominant protection against the edit regardless of whether the edit is located in cis or trans. This could be achieved by including in the construct a module which nullifies the effects of the edit at the RNA level, for example an RNA editor which restores the RNA expressed from the edited gene back to the WT seq or an RNAi module which degrades the edited RNA.

In the absence of the construct, the edit should cause dominant lethality or sterility in both sexes (strategy 1 [bB-]), only females (strategy 3 [bF-]), in females with recessive lethality/sterility in males (strategy 4 [bFm-]) or in males with recessive lethality/sterility in females (strategy 2 [bfM-]). FIG. 8 shows time courses for the relative female population size after a single release of these constructs in male heterozygotes at 50% of the initial male population, where the design which achieves suppression the fastest involves edits in both sexes (strategy 1 [bB-]).

FIG. 10a illustrates an example single locus molecular design for strategy 1 of FIG. 8. Here the construct (shown here as Cas9 and gRNA) is inserted into a haplo-sufficient gene required in both sexes (HS^{ETDN}, grey). The arrows indicate that in organisms that are heterozygous for the genomic editor and a wild type allele of the HS gene, the editor acts on a wild type target sequence on the chromosome without the construct to produce a dominant negative mutation that is lethal or sterile for both males and females (orange arrow); the target site on the chromosome with the construct has been recoded such that it is still functional but not recognised by the editor (rHS^{ETDN}; no orange arrow); The construct also encodes and RNAeditor (RNAe) or RNA interference (RNAi) module which acts to nullify the effects of the editor and provide dominant rescue. In a wild type genetic background, individuals carrying one or more edited target sites are lethal or sterile. In individuals carrying at least one copy of the edited target site and a construct, the edited RNA transcribed from the gene carrying the edited allele (grey line with edit shown in orange) is either degraded (through the action of the RNAi module encoded in the construct) or is reverted back to wild-type (grey line, through the action of the RNA editor encoded in the construct) before the RNA is translated, protecting the individual against the effects of the dominant negative mutation. The RNA transcribed from the chromosome carrying the recoded copy of the target gene (grey line with recoded sequence shown in grey) will be resistant to the activity of the RNAe or RNAi module. Note that here (and in all other constructs containing and RNAe/i module described below) if the construct encodes an RNA editor it is not necessary for the target site on the chromosome with the construct to be recoded since the RNA transcribed from it will also be reverted back to the wild-type. Strategies 2-4 of FIG. 8 can be built with a similar molecular configuration as shown in FIG. 10a, where, for example, the target site is located in a sex-specific intron. Alternative 2-locus molecular designs are also possible for strategies 1-4, where, for example, the target site of the genomic editor is located in a separate gene, either near to the disrupted gene (<1% meiotic recombination) or distant from the disrupted gene (>1% meiotic recombination).

Improvements in efficiency can be made if the construct is associated with female-specific recessive lethality or sterility, provides dominant protection against the edit it creates regardless of its location relative to the integration site of the construct (e.g. via RNA editing or RNA interference) and the genomic editor is designed to create either bi-sex dominant edits (strategy 5 [fB-]) or female-specific edits (strategiy 9 [fF-]). Although the same level of equilibrium suppression is achieved, it takes longer to be reached if editing occurs in only one sex (strategy 6 and 7), the edit is bisex recessive (strategy 8) or the edit results in some male costs (strategy 10-13).

FIGS. 10b and c show example molecular designs for strategies 9 and 5 of FIG. 8 respectively which involve a single gene. As illustrated in FIG. 10b (left), one way of achieving strategy 9 of FIG. 8 is for the genetic construct to integrate into and disrupt a female-specific haplo-sufficient gene (HS^{ETDN}, pink). Alternatively, the genetic construct integrates into and disrupts a female-specific exon of a haplo-sufficient gene needed in both sexes and the genomic editor generates either sex-specific (FIG. 10b [right]; HS^{ETDN}, pink; strategy 9) or bi-sex (FIG. 10c [left]; HS^{ETDN}, grey; strategy 5) dominant negative or gain of function mutations. As shown in FIG. 10c (right) an alternative way to achieve strategy 5 is for the genetic construct to integrate into and disrupt a bi-sex haplo-sufficient gene (HS^{ETDN}, grey) and to contain sequences that ensure the construct is spliced out in a sex-specific manner. Again, thin pink and blue lines show the sex-specific splicing pattern of the gene; for simplicity, they are only shown on the top chromosome, but also apply to the bottom (wild type) allele. In each case the constructs must also function to nullify the effects of the editor and provide dominant rescue (RNAe/i) and the target site on the chromosome with the construct should be recoded such that it is still functional but not recognised by the editor (rHS^{ETDN}). Strategies 6-8 and 10-13 of FIG. 8 can be built with a similar molecular configuration as shown in FIGS. 10b and c, where, for example, editing occurs in only one sex and/or the sex-specificity of the edited allele differs from that in strategy 5 or 9.

Alternatively, strategies 5-13 can be implemented using two tightly linked genes. FIGS. 10e and f show example 2-locus molecular designs for strategies 9 and 5 of FIG. 8 respectively where the genomic editor acts on a wild type allele of another gene that is near to the disrupted gene. One way of achieving strategy 5 and 9 of FIG. 8 is for the genetic construct to integrate into and disrupt a female-specific haplo-sufficient gene (HS, pink) (FIG. 10e [left] and FIG. 10f [left]) or for the genetic construct to integrate into and disrupt a bi-sex haplo-sufficient gene and to contain sequences that ensure the construct is spliced out in a sex-specific manner (FIG. 10e [right] and FIG. 10f [right]). Again, in each case the constructs may also function to nullify the effects of the editor and provide dominant rescue (RNAe/i), and the target site on the chromosome with the construct has been recoded such that it is still functional but not recognised by the editor (rHI/rHS^{ETDN}). Strategies 6-8 and 10-13 of FIG. 8 can be built with a similar molecular configuration as shown in FIGS. 10b and c, where, for example, editing occurs in only one sex and/or the sex-specificity of the edited allele differs from that in strategy 5 or 9.

Alternatively, if the construct is sex-specific and the genomic editor produces dominant edits which effect the opposite sex (FIG. 8. strategies 14 [fMM], 18 [ffMM], 22 [mFF] and 23 [mFmF]), partial suppression can be achieved without the need for the construct to confer dominant protection against the edits (i.e. contain an RNA editing or interference module). FIG. 10d illustrates an example molecular configuration for strategy 22 (fMM) using a single gene which has both male and female sex-specific exons, for example doublesex. Here the construct is inserted into and disrupts a male-specific exon (HS, blue) such that the construct is spliced out in females and therefore only male homozygotes are lethal or sterile. The genomic editor is configured to create dominant mutations in a female-specific exon (HS^{ETDN}, pink), where in a wild type background males are sterile or lethal. The construct must also provide protection to the target site located on the same the chromosome, for example by modifying or removing the target site such that it is no longer recognised by the editor (rHS^{ETDN}). Strategy 14 can be implemented in a similar way where the sexes in the example shown in FIG. 10d are reversed. FIG. 10g illustrates an example 2-locus molecular design for strategy 14 where the genomic editor acts on a wild type allele of another gene that is tightly linked to the disrupted gene. Here the genetic construct integrates into and disrupts a female-specific haplo-sufficient gene (HS, pink; left) or a bi-sex haplo-sufficient gene (HS, grey; right) and contains sequences that ensure the construct is spliced out in a sex-specific manner. The editor could create dominant knock-out mutations in a male-specific haplo-insufficient gene (HI, blue) or create dominant negative mutations in any gene where this is possible (HS^{ETDN}, blue). Again, the construct may also provide protection to the target site located on the same the chromosome. Where the editor creates a knock-out of a HI gene, this may be a recoded version of the gene which is still functional but not recognised by the editor (rHI), whereas if the edit is a dominant negative this could simply involve removing the gene. Strategy 18, 22 and 23 can be implemented using a similar molecular design as illustrated for strategy 14, where the sex-specificity of the genes and/or splicing differ from that of strategy 14.

For each design, increased levels of suppression could be achieved if, after suppression has been achieved by the first release, a second construct of the same design located elsewhere in the genome is released into the same population. This is demonstrated for the most efficient six designs in FIGS. 8c and d.

### 3.2 2-Locus Screen

Eight strategies are found in the 2-locus screen, both when simulated with idealised and non-idealised parameters. Two strategies (22 [mFFB] and 14 [fMMB]) were common between the 1- and 2-locus screens. FIG. 10h illustrates example 2-locus molecular designs for strategy 14 of FIG. 8 where the genomic editor acts on a wild type allele of another gene that is distant from the disrupted gene. Here the genetic construct integrates into and disrupts a sex-specific haplo-sufficient gene (FIG. 10h [left]) or a bi-sex haplo-sufficient gene and contains sequences that ensure the construct is spliced out in a sex-specific manner (FIG. 10h). In each case the construct may also encode a recoded version of the target gene such that it is resistant to editing whilst also restores function in individuals carrying a single copy of the dominant edit (rHI/rGene^{ETDN}). For strategies 14 and 22, if the genomic editor acts on a wild type allele of another unlinked gene, the strategy will still work if editing occurs in only one sex (indicated with a blue arrow and the initial of the sex in which expression occurs), though with somewhat different dynamics (FIG. 9, strategies 27, 29, 31 and 32).

Finally the 2-locus screen revealed two additional strategies (28 and 30) where the construct is sex-specific and the genomic editor creates an edit which affects the opposite sex to that affected by the disruption. This could be achieved by expressing Caso from a promoter only active in a single sex. FIG. 10i illustrates example 2-locus molecular designs for strategy 28. Again, the construct may encode a recoded version of the target gene in order to restore function in individuals carrying a single copy of the dominant edit (rHI/rGene^{ETDN}).

### CONCLUSIONS

The inventors have identified a novel genetic construct that can disrupt a haplo-sufficient gene needed for survival or reproduction in male and/or female organisms. Additionally, the genetic construct encodes a genomic editor that creates a dominant lethal or sterile phenotype in males and/or females, such that individuals with the mutation are unable to reproduce. Advantageously, therefore, the genetic construct can be inserted into an organism and released into a population, in order to suppress the population in an efficient manner over a period of time.

In summary, the genetic construct according to the invention differs from previous genetic control elements for controlling pest populations, in that:
The gRNA and Cas9 are designed to induce dominant mutations at a locus outside of the insertion site of the construct. This may or may not be within a haplosufficient gene.
The construct is designed such that it provides protection against a single copy of the dominant mutations it creates.
The gRNA and Cas9 in the claimed genetic construct does not result in homing of the construct and population suppression is expected to be achievable without homing.

The inventors believe they are the first to design a genetic construct which: (i) causes recessive sterility or lethality, (ii) creates dominant mutations, and (iii) protects against the dominant mutations.

Advantageously, the inventors discovered that depending on the precise configuration, the genetic construct can have three different types of potentially useful impact when released into a target population: localised suppression, non-localised partial suppression, or non-localised complete suppression.

1. A genetic construct comprising a first nucleotide sequence configured to disrupt a haplo-sufficient gene needed for survival or reproduction in a male and/or female organism, or to integrate into or near to a disrupted allele of the haplo-sufficient gene, and a second nucleotide sequence encoding a genomic editor that creates a dominant lethal or sterile mutation in a target gene expressed in the male and/or female organism, such that a homozygote for the genetic construct is lethal or sterile and/or a heterozygote for the dominant lethal or sterile mutation is lethal or sterile.

2. The genetic construct according to claim 1, wherein the genetic construct is configured to disrupt the haplo-sufficient gene via integration of the genetic construct into the haplo-sufficient gene at an integration site or disruption site, optionally wherein the genetic construct introduces a premature stop codon into the haplo-sufficient gene.

3. The genetic construct according to claim 2, wherein the genetic construct integrates into a region of the haplo-sufficient gene between its 5′ promoter and 3′ terminus, or wherein the genetic construct integrates between exons or introns of the haplo-sufficient gene.

4. The genetic construct according to claim 1, wherein the genetic construct is configured to integrate into the disrupted allele of the haplo-sufficient gene, or at a location outside of, but near to, the disrupted allele of the haplo-sufficient gene, preferably within 10, 8, 6, 4, 2, or 1 centiMorgan of the haplo-sufficient gene, optionally wherein the haplo-sufficient gene is disrupted by the introduction of a knock-out mutation, optionally by the introduction of a premature stop codon, or by deletion of all or part of the haplo-sufficient gene.

5. The genetic construct according to any preceding claim, wherein the genetic construct is configured to disrupt a haplo-sufficient gene needed for survival or reproduction in a male and female organism, or to integrate into or near to a disrupted allele of the haplo-sufficient gene, such that male and female homozygotes for the genetic construct are lethal or sterile.

6. The genetic construct according to any preceding claim, wherein the genomic editor creates a dominant lethal or sterile mutation in a target gene expressed in the female organism, or in a target gene expressed in the male and female organism.

7. The genetic construct according to any preceding claim, wherein the target gene is a wild type allele of the disrupted haplo-sufficient gene.

8. The genetic construct according to any preceding claim, wherein the genomic editor creates a dominant negative or a dominant gain of function mutation in a wild type allele of the disrupted haplo-sufficient gene.

9. The genetic construct according to either claim 7 or 8, wherein the genomic editor targets a site in the haplo-sufficient gene that is downstream of the integration site, or wherein the genomic editor targets a site in the haplo-sufficient gene that is upstream of the integration site.

10. The genetic construct according to any preceding claim, wherein the genomic editor creates a dominant negative or a dominant gain of function mutation in a female-specific exon of a haplo-sufficient gene needed in a male and female organism, optionally wherein the haplo-sufficient gene is a homolog of the Drosophila gene doublesex or fruitless.

11. The genetic construct according to any one of claims 1-6, wherein the target gene is a gene that is located near to the disrupted haplo-sufficient gene.

12. The genetic construct according to claim 11, wherein the genomic editor creates a dominant negative or a dominant gain of function mutation in a wild type allele of a target gene that is located near to the disrupted haplo-sufficient gene, or wherein the genomic editor creates a knock-out mutation in a wildtype allele of a haplo-insufficient target gene that is located near to the disrupted haplo-sufficient gene.

13. The genetic construct according to either claim 11 or 12, wherein the target gene and the disrupted haplo-sufficient gene comprise a meiotic recombination fraction of less than 5%, less than 4%, less than 3%, less than 2%, or less than 1%.

14. The genetic construct according to any preceding claim, wherein the target site on the chromosome containing the construct is modified or removed, such that it is not recognised by the genomic editor.

15. The genetic construct according to any one of claims 1-6, wherein the target gene is a gene that is distantly located from the disrupted haplo-sufficient gene.

16. The genetic construct according to claim 15, wherein the genomic editor creates a dominant negative or a dominant gain of function mutation in a wild type allele of a target gene that is distantly located from the disrupted haplo-sufficient gene, or wherein the genomic editor creates a knock-out mutation in a wild type allele of a haplo-insufficient target gene that is distantly located from the disrupted haplo-sufficient gene.

17. The genetic construct according to either claim 15 or 16, wherein the target gene and the disrupted haplo-sufficient gene comprise a meiotic recombination fraction of greater than 0.5%, greater than 1%, greater than 2%, greater than 3%, greater than 4%, or greater than 5%.

18. The genetic construct according to any preceding claim, wherein the genetic construct further comprises a nucleotide sequence encoding a rescue copy of the target gene.

19. The genetic construct according to any one of claims 1-4, wherein the genetic construct is configured to disrupt a haplo-sufficient gene needed for survival or reproduction in a male or female organism, or to integrate into or near to a disrupted allele of the haplo-sufficient gene, such that male or female homozygotes for the genetic construct are lethal or sterile.

20. The genetic construct according to claim 19, wherein the genetic construct comprises a nucleotide sequence encoding a genomic editor that creates a dominant lethal or sterile mutation in a target gene expressed in the male and/or female organism.

21. The genetic construct according to claim 19 or 20, wherein the target gene is a wild type allele of the disrupted haplo-sufficient gene.

22. The genetic construct according to any one of claims 19-21, wherein the genomic editor creates a dominant negative or a dominant gain of function mutation in a wild type allele of the disrupted haplo-sufficient gene, or wherein the genomic editor creates a dominant negative or a dominant gain of function mutation in a wild type allele of a target gene that is located near to the disrupted haplo-sufficient gene, or wherein the genomic editor creates a knock-out mutation in a wild type allele of a haplo-insufficient target gene that is located near to the disrupted haplo-sufficient gene.

23. The genetic construct according to any one of claims 19-22, wherein the genomic editor creates a dominant negative mutation or a dominant gain of function mutation in a female-specific haplo-sufficient gene, optionally wherein the female-specific haplo-sufficient gene is selected from homologs of the Drosophila genes ovo, dorsal, torso, easter, and Toll.

24. The genetic construct according to any one of claims 19-22, wherein the genomic editor creates a dominant negative mutation or a dominant gain of function mutation in a male-specific haplo-sufficient gene, optionally wherein the male-specific haplo-sufficient gene is selected from homologs of the Drosophila genes betaTub85D and whirligig.

25. The genetic construct according to any one of claims 19-22, wherein the disrupted haplo-sufficient gene is disrupted at a female-specific exon of the haplo-sufficient gene, optionally wherein the disrupted haplo-sufficient gene is disrupted at a female-specific exon of the doublesex or fruitless genes, or a female-specific exon of a homolog of the doublesex or fruitless genes.

26. The genetic construct according to any one of claims 19-22, wherein the disrupted haplo-sufficient gene is disrupted at a male-specific exon of the haplo-sufficient gene, optionally wherein the disrupted haplo-sufficient gene is disrupted at a male-specific exon of the doublesex or transformer genes, or a male-specific exon of a homolog of the doublesex or transformer genes.

27. The genetic construct according to any one of claims 19-26, wherein the genetic construct comprises a nucleotide sequence that ensures the construct will be spliced out at the RNA in a male organism, optionally wherein the genetic construct comprises a nucleotide sequence encoding a splicing control sequence of a homolog of a transformer, doublesex or fruitless gene, that ensures the construct will be spliced out at the RNA in a male organism.

28. The genetic construct according to any one of claims 19-26, wherein the genetic construct comprises a nucleotide sequence that ensures the construct will be spliced out at the RNA in a female organism, optionally wherein the genetic construct comprises a nucleotide sequence encoding a splicing control sequence of a homolog of a Drosophila transformer gene, that ensures the construct will be spliced out at the RNA in a female organism.

29. The genetic construct according to any one of claims 19-28, wherein the target site on the chromosome containing the construct is modified or removed, such that it is not recognised by the genomic editor.

30. The genetic construct according to claim 29, wherein the chromosome comprising the genetic construct is protected from the genomic editor because the target site has been recoded, and expression of the recoded target gene is enhanced in a sex-specific manner, or the chromosome comprising the genetic construct contains a second copy of the recoded target gene with control sequences that ensure it is only expressed in a male or female, such that only a male or female heterozygote comprising the construct and the mutation is affected.

31. The genetic construct according to any one of claims 1-4, wherein the genetic construct comprises a nucleotide sequence encoding an RNA editor or an RNA interference module, preferably wherein the RNA editor or RNA interference module restores RNA expressed from the mutated gene back to the wild type sequence, by RNA editing, or removes the RNA transcribed from the edited gene, by RNA interference (RNAi).

32. The genetic construct according to claim 31, wherein the RNA editor or the RNAi module are expressed only in one sex.

33. The genetic construct according to any one of claims 1-4, wherein the genetic construct is configured to disrupt a haplo-sufficient gene essential for fertility or viability in either a male or female organism, or to integrate into or near to a disrupted allele of the haplo-sufficient gene, and wherein the genomic editor produces a dominant negative or a dominant gain of function mutation in a wild type allele of the same locus that affects the opposite sex to the gene disruption.

34. The genetic construct according to claim 33, wherein the disrupted haplo-sufficient gene is disrupted at a male-specific exon causing recessive male-specific lethality or sterility, and wherein the genomic editor creates a dominant sterile mutation in a female-specific exon, optionally wherein the disrupted and target gene is a homolog of doublesex.

35. The genetic construct according to claim 33, wherein the disrupted haplo-sufficient gene is disrupted at a female-specific exon causing recessive female-specific lethality or sterility, and wherein the genomic editor creates a dominant sterile mutation in a male-specific exon, optionally wherein the disrupted and target gene is a homolog of doublesex.

36. The genetic construct according to any one of claims 1-4, wherein the genetic construct is configured to disrupt a haplo-sufficient gene essential for fertility or viability in either a male or female organism, or to integrate into or near to a disrupted allele of the haplo-sufficient gene, and wherein the genomic editor targets a wild type allele of a target gene that is located near to the disrupted haplo-sufficient gene, to produce a dominant negative mutation, a gain of function mutation, or a knock-out of a haplo-insufficient gene.

37. The genetic construct according to any one of claims 1-4, wherein the genetic construct is configured to disrupt a haplo-sufficient gene essential for fertility or viability in either a male or female organism, or to integrate into or near to a disrupted allele of the haplo-sufficient gene, and wherein the genomic editor targets a wild type allele of a gene that is located near to the disrupted haplo-sufficient gene, to produce a dominant negative mutation, a gain of function mutation, or a knock-out of a haplo-insufficient gene that affects the opposite sex to that affected by the gene disruption.

38. The genetic construct according to any one of claims 31-37, wherein the target site on the chromosome containing the construct is modified or removed, such that it is not recognised by the genomic editor, or wherein the genetic construct comprises a nucleotide sequence that ensures it will be spliced out in a sex-specific manner.

39. The genetic construct according to any one of claims 1-4, wherein the genetic construct is configured to disrupt a haplo-sufficient gene essential for fertility or viability in either a male or female organism, or to integrate into or near to a disrupted allele of the haplo-sufficient gene, and wherein the genomic editor targets a wild type allele of a gene that is distantly located from the disrupted haplo-sufficient gene, to produce a dominant negative mutation, a gain of function mutation, or a knock-out of a haplo-insufficient gene that affects the opposite sex to that affected by the gene disruption.

40. A genetic construct comprising a first nucleotide sequence configured to integrate into a location outside of a haplo-sufficient gene needed for survival or reproduction in a male and/or female organism, and a second nucleotide sequence encoding: (i) a first genomic editor that creates a dominant lethal or sterile mutation in a target gene expressed in the male and/or female organism, such that a heterozygote for the dominant lethal or sterile mutation is lethal or sterile; and (ii) a second genomic editor that creates a recessive lethal or sterile mutation in the haplo-sufficient gene, such that a homozygote for the recessive lethal or sterile mutation is lethal or sterile.

41. The genetic construct according to claim 40, wherein the second genomic editor creates a premature stop codon in the haplo-sufficient gene.

42. The genetic construct according to claim 40 or claim 41, wherein the first genomic editor creates a dominant lethal or sterile mutation in a haplo-insufficient gene or any gene in which it is possible to create dominant lethal or sterile mutations in males and females, or in females only.

43. The genetic construct according to any one of claims 40 to 42, wherein the genetic construct further comprises a nucleotide sequence encoding a rescue copy of the target gene.

44. The genetic construct according to any preceding claim, wherein the organism is selected from the group consisting of: disease vectors, agricultural pests or undesired invasive species, optionally wherein the types of pest species include arthropods, other invertebrates, mammals, other vertebrates and weeds.

45. The genetic construct according to claim 44, wherein the arthropod is an insect, arachnid, myriapod or crustacean, optionally wherein the insect is a mosquito, a tephritid fruit fly, a sandfly, a dipteran, a lepidopteran, a coleopteran, or a mealybug.

46. The genetic construct according to claim 44, wherein the organism is a gastropod, a bivalve, a fish, an amphibian, a mammal, or a plant, preferably a weed.

47. The genetic construct according to any preceding claim, wherein the target gene is a wild type allele of the disrupted haplo-sufficient gene, or wherein the target gene is any other gene where a dominant negative or a dominant gain of function mutation can be produced.

48. The genetic construct according to any preceding claim, wherein the target gene encodes a protein that acts as a multimer, or wherein the target gene encodes a transcription factor or a membrane-bound protein, preferably a homodimeric membrane receptor.

49. The genetic construct according to any preceding claim, wherein the target gene is a haplo-insufficient gene, optionally wherein the haplo-insufficient gene is a haplolethal gene.

50. The genetic construct according to any preceding claim, wherein the genomic editor is selected from a group consisting of: a transcription activator-like effector nuclease (TALEN) genomic editor; Zinc finger nuclease (ZFN) genomic editor; and a CRISPR-based genomic editor, preferably wherein the genomic editor is a CRISPR-based genomic editor, most preferably a CRISPR-Cpf1-based or CRISPR-Cas9-based genomic editor.

51. The genetic construct according to any preceding claim, wherein the genomic editor comprises a first nucleotide sequence that is capable of hybridising to the target gene, preferably wherein the first nucleotide sequence which is capable of hybridising to the target gene is a guide RNA (gRNA).

52. The genetic construct according to claim 51, wherein the genomic editor further comprises a second nucleotide sequence encoding a CRISPR nuclease, preferably a Cpf1 or Cas9 nuclease, and most preferably a Cas9 nuclease, or a derivative thereof to allow for DNA nicking, base editing, prime editing, or other types of edit.

53. The genetic construct according to claim 52, wherein the genomic editor further comprises at least one promoter sequence, which drives expression of the first and second nucleotide sequence, or wherein the genomic editor comprises a first promoter sequence operably linked to the first nucleotide sequence and a second promoter sequence operably linked to the second nucleotide sequence.

54. The genetic construct according to any preceding claim, wherein the genomic editor further comprises a nucleotide sequence comprising a control sequence that ensures the genomic editor is active in a male and/or female germline, optionally wherein the genetic construct comprises a first and second nucleotide sequence comprising a first and second control sequence.

55. The use of the genetic construct according to any one of claims 1-54, to disrupt a haplo-sufficient gene needed for survival or reproduction in a male and/or female organism, or to integrate into or near to a disrupted allele of the haplo-sufficient gene, such that a homozygote for the genetic construct is lethal or sterile, and to create a dominant lethal or sterile mutation in a target gene expressed in a male and/or female organism, such that a heterozygote for the dominant lethal or sterile mutation is lethal or sterile, and/or a male and/or female organism comprising the mutation is unable to reproduce.

56. The use of the genetic construct according to any one of claims 40 to 43, to integrate into a location outside of a haplo-sufficient gene needed for survival or reproduction in a male and/or female organism, and to create a dominant lethal or sterile mutation in a target gene expressed in the male and/or female organism, such that a heterozygote for the dominant lethal or sterile mutation is lethal or sterile, and to create a recessive lethal or sterile mutation in the haplo-sufficient gene expressed in the male and/or female organism, such that a homozygote for the recessive lethal or sterile mutation is lethal or sterile.

57. A method of producing a genetically modified organism, the method comprising introducing, into an organism, the genetic construct according to any one of claims 1-54.

58. A method of producing a genetically modified organism, the method comprising introducing, into an organism, a genetic construct comprising a first nucleotide sequence configured to disrupt a haplo-sufficient gene needed for survival or reproduction in a male and/or female organism, or to integrate into or near to a disrupted allele of the haplo-sufficient gene, and a second nucleotide sequence encoding a genomic editor that creates a dominant lethal or sterile mutation in a target gene of the male and/or female organism, such that a homozygote for the genetic construct is lethal or sterile and/or a heterozygote for the dominant lethal or sterile mutation is lethal or sterile.

59. A method of producing a genetically modified organism, the method comprising introducing, into an organism, a genetic construct comprising a first nucleotide sequence configured to integrate into a location outside of a haplo-sufficient gene needed for survival or reproduction in a male and/or female organism, and a second nucleotide sequence encoding: (i) a first genomic editor that creates a dominant lethal or sterile mutation in a target gene expressed in the male and/or female organism; and (ii) a second genomic editor that creates a recessive lethal or sterile mutation in the haplo-sufficient gene, such that a homozygote for the recessive lethal or sterile mutation is lethal or sterile.

60. The method according to claim 57 or 58, further comprising introducing into the organism a second genetic construct comprising a nucleotide sequence configured to increase the frequency of the first genetic construct, wherein the first genetic construct is the genetic construct that disrupts, or is associated with a disruption of, the haplo-sufficient gene.

61. The method according to claim 60, wherein the second genetic construct comprises a nucleotide sequence encoding a guide RNA that targets the integration site of the first genetic construct, or wherein the second genetic construct comprises a first nucleotide sequence encoding a first guide RNA that targets the integration site of the first genetic construct, and a second nucleotide sequence encoding a second guide RNA that targets the integration site of the second genetic construct.

62. A genetically modified organism obtained or obtainable by the method according to any one of claims 57-61.

63. A genetically modified organism comprising a disrupted haplo-sufficient gene needed for survival or reproduction in a male and/or female organism, and a nucleotide sequence encoding a genomic editor that creates a dominant lethal or sterile mutation in a target gene, such that the male and/or female organism comprising the mutation is unable to reproduce.

64. A genetically modified organism comprising a disrupted haplo-sufficient gene needed for survival or reproduction in a male and/or female organism, and a nucleotide sequence encoding a first genomic editor that creates a dominant lethal or sterile mutation in a target gene expressed in the male and/or female organism, such that a heterozygote for the dominant lethal or sterile mutation is lethal or sterile, and a second genomic editor that creates a recessive lethal or sterile mutation in the haplo-sufficient gene expressed in the male and/or female organism, such that a homozygote for the recessive lethal or sterile mutation is lethal or sterile.

65. A method of suppressing a wild type population of an organism, the method comprising breeding a genetically modified organism comprising a genetic construct comprising a first nucleotide sequence configured to disrupt a haplo-sufficient gene needed for survival or reproduction in a male and/or female organism, or to integrate into or near to a disrupted allele of the haplo-sufficient gene, and a second nucleotide sequence encoding a genomic editor that creates a dominant lethal or sterile mutation in a target gene of the male and/or female organism, such that a homozygote for the genetic construct is lethal or sterile and/or a heterozygote for the dominant lethal or sterile mutation is lethal or sterile.

66. A method of suppressing a wild type population of an organism, the method comprising breeding a genetically modified organism comprising a genetic construct comprising a first nucleotide sequence configured to integrate into a location outside of a haplo-sufficient gene needed for survival or reproduction in a male and/or female organism, and a second nucleotide sequence encoding: (i) a first genomic editor that creates a dominant lethal or sterile mutation in a target gene expressed in the male and/or female organism; and (ii) a second genomic editor that creates a recessive lethal or sterile mutation in the haplo-sufficient gene expressed in the male and/or female organism, such that a homozygote for the recessive lethal or sterile mutation is lethal or sterile.

67. The use of a genetic construct according to any one of claims 1-54, to suppress a wild-type population of an organism.

68. The method according to claim 65 or 66, or the use according to claim 67, wherein the suppression is localised suppression, non-localised partial suppression, or non-localised complete suppression.
